(19) United States
(12) Patent Application Publication
(10) Pub. No.: US 2008/0199419 A1
(43) Pub. Date: Aug. 21, 2008

(54) Use of Water-Soluble Polymers Which are Obtained by Means of Controlled Radical Polymerisation as an Agent For Improving the Opacity and/or Brightness of Dry Products Containing Same

(75) Inventors: Jean-Marc Suau, Lucenay (FR); Christian Jacquemet, Lyon (FR); Jacques Mongoin, Quincieux (FR)

Correspondence Address: OBLON, SPIVAK, MCCLELLAND MAIER & NEUSTADT, P.C., 1940 DUKE STREET, ALEXANDRIA, VA, 22314, US

(73) Assignee: Coatex S.A.S., Genay (FR)

(21) Appl. No.: 11/913,948

(22) PCT Filed: May 12, 2006
(86) PCT No.: PCT/FR06/01064

§ 371 Date: Nov. 9, 2007

(30) Foreign Application Priority Data
May 20, 2005 (FR) 05/05054

(51) Int. Cl. A61K 8/72; C08F 20/00; B05D 3/00

(52) U.S. Cl. 424/78.02; 524/556; 427/385.5

(57) ABSTRACT

The invention relates to agents that are used to improve optical properties such as opacity and/or brightness of dry products based on mineral matter containing said agents. More specifically, the invention relates to a method of producing dry products based on mineral matter, in which the agents used to improve the opacity and/or brightness of said dry products take the form of controlled-structure water-soluble polymers which are obtained using a controlled radical polymerisation method employing a particular alkoxyamine as a polymerisation initiator. The invention also relates to the use of aqueous suspensions and/or dispersions of mineral matter containing said polymers for the same purpose. The aforementioned dry products comprise plastics, filled paper, dry films produced by drying a coating slip, dry films produced by drying an aqueous paint formulation or dry films produced by drying an aqueous cosmetic formulation, which contain mineral matter as well as said polymers.

The present invention concerns the sector of agents enabling the improvement of optical properties, such as notably opacity and/or brightness, of mineral matter-based dry products containing the said agents.

The invention firstly concerns the use in a manufacturing process of mineral matter-based dry products, as agents improving opacity and/or brightness of the said dry products, of hydrosoluble polymers of controlled structure obtained by a process of controlled radical polymerisation using, as the polymerisation initiator, a particular alcoxyamine of general formula (A):

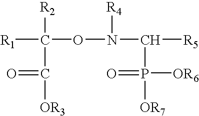

where:
R₁ and R₂ represent an alkyl radical, linear or branched, with a number of carbon atoms ranging from 1 to 5,
R₃ represents a hydrogen atom, an alkyl radical, linear or branched, with a number of carbon atoms ranging from 1 to 8, a phenyl radical, a cation such as L+, Na+, K+, H4N+, Bu3HN⁺, where Bu=butyl,
R represents an alkyl radical, linear or branched, with a number of carbon atoms ranging from 1 to 8, and preferentially a tertiobutyl radical,
R₅ represents an alkyl radical, linear or branched, with a number of carbon atoms ranging from 1 to 8, and preferentially a tertiobutyl radical,
R₆ and R₇ represent an alkyl radical, linear or branched, with a number of carbon atoms ranging from 1 to 8, and preferentially an ethyl radical,

In a first variant, this use of the said hydrosoluble polymers is characterised in that the manufacturing process of the said dry products includes at least one stage of grinding and/or at least one stage of dispersion and/or at least one stage of concentration in water of the mineral matter, which leads to a dispersion and/or aqueous suspension of mineral matter being obtained.

The dispersion and/or aqueous suspension of the mineral matter thus obtained enables the manufacture of water-based formulations of mineral matter, in the fields of paper and notably in paper coatings and in mass filler, in water-based paints, and in cosmetic compositions.

The said formulations then allow, after drying, dry products to be obtained, the opacity and/or brightness of which is improved. These dry products are papers filled by the previous mass fillers, or dry coverings which are dry films resulting from the drying of a paper coating, dry films resulting from the drying of a water-based paint formulation, or dry films resulting from the drying of a water-based cosmetic formulation.

Moreover, the dispersion and/or water-based suspension of mineral matter previously obtained may be dried; the invention then concerns also the dry powders thus obtained. The said dry powders then allow the manufacture of plastics the opacity and/or brightness of which is improved. The said dry powders also enable water-based formulations of mineral matter to be manufactured, and notably paper coatings, mass fillers, water-based paints and cosmetic formulations. These said formulations then allow, after drying, dry products to be obtained, the opacity and/or brightness of which is improved. These dry products are papers filled by the previous mass fillers, or dry coverings which are dry films resulting from the drying of a paper coating, dry films resulting from the drying of a water-based paint formulation, or dry films resulting from the drying of a water-based cosmetic formulation.

In a second variant, this use in a process of manufacture of mineral matter-based dry products is characterised in that the said polymers are used as direct additives in the field of plastics, and also in the field of water-based formulations, and notably in the paper sectors, and particularly in paper coatings, in water-based paint formulations and in cosmetic formulations.

The plastics then obtained have a high opacity and/or brightness. Moreover, the said previously obtained formulations then allow, after drying, dry products to be obtained, the opacity and/or brightness of which is improved. These dry products are dry coverings which are dry films resulting from the drying of a paper coating, dry films resulting from the drying of a water-based paint formulation, or dry films resulting from the drying of a water-based cosmetic formulation.

Secondly, the invention concerns the use of a dispersion and/or a water-based suspension of mineral matter containing the said polymers, in a process to manufacture dry products containing mineral matter, as agents improving the opacity and/or brightness of the said dry products, in the fields of paper, and notably in paper coatings, and in mass fillers, in water-based paints, and in cosmetic compositions.

The dispersion and/or aqueous suspension of the mineral matter thus obtained enables the manufacture of water-based formulations of mineral matter, in the fields of paper and notably in paper coatings and in mass filler, in water-based paints, and in cosmetic compositions.

The said formulations then allow, after drying, dry products to be obtained, the opacity and/or brightness of which is improved. These dry products are papers filled by the previous mass fillers, or dry coverings which are dry films resulting from the drying of a paper coating, dry films resulting from the drying of a water-based paint formulation, or dry films resulting from the drying of a water-based cosmetic formulation.

Moreover, the dispersion and/or water-based suspension of mineral matter previously obtained may be dried, and the dry powders thus obtained then allow the manufacture of plastics, the opacity and/or brightness of which is improved. The said dry powders also enable water-based formulations of mineral matter to be manufactured, and notably paper coatings, mass fillers, water-based paints and cosmetic formulations. The said formulations then allow, after drying, dry products to be obtained, the opacity and/or brightness of which is improved. These dry products are papers filled by the previous mass fillers, or dry coverings which are dry films resulting from the drying of a paper coating, dry films resulting from the drying of a water-based paint formulation, or dry films resulting from the drying of a water-based cosmetic formulation.

The invention also concerns dispersions and water-based suspensions of mineral matter, water-based formulations of mineral matter, dry mineral matter powders, dry mineral matter products which cover both plastics and filled papers, together with the dry coverings obtained according to the abovementioned methods, i.e. according to the invention.

By the expression “dispersion and/or water-based suspension of mineral matter”, the Applicant means a product containing:
water,
at least one mineral matter,
possibly at least one dispersing agent and/or at least one grinding aid agent,
and possibly at least one polymer, use of which is made in the invention.

The term dispersion designates the case of the previous composition containing at least one dispersing agent; the said dispersion results from a stage of dispersion proper, or from a stage of concentration, with use of a dispersing agent.

The term suspension designates the case of the previous composition containing at least one grinding aid agent, where the said grinding aid agent facilitates the mechanical grinding action which causes the size of the particles to be reduced.

The expression “water-based mineral matter formulations” designates a composition containing:
a dispersion and/or a water-based suspension of mineral matter, as defined above,
one or more different constituents of a grinding aid agent and a dispersing agent; as non-restrictive examples, these constituents in question may be, in the context of a paper coating, a binder used in the paper field, an optical brightener, a thickening agent, or in the context of a water-based paint, a binder used in the field of water-based paints, a coalescence agent, a rheology modifier, or again it may be an anti-foaming agent called a surface active agent, a biocide, or other.

Furthermore, the ter “paints” also designates, in a non-restrictive manner, inks, treatment varnishes, or thick or semi-thick plastic coverings, with both gloss and satin finishes. In addition, apart from the mineral matter used to manufacture the said paints in the context of the invention, the skilled man in the art may also incorporate into them organic pigments, which may be dispersed through the use of polymers according to the invention, such as, for example, carbon black, phthalocyanine derivatives and azo derivatives, or their mixtures.

The said water-based formulations of mineral matter allow the manufacture of “dry products”, which are in fact:
either filled papers containing a mass filler manufactured according to invention,
or “dry coverings” resulting from the drying of the said formulations (which have been previously applied to a support) and which may be, in the field of this invention: dry films resulting from the drying of a paper coating, dry films resulting from the drying of a water-based paint formulation, or dry films resulting from the drying of a water-based cosmetic formulation.

In this process, the water-based formulations in question are, clearly, previously applied on supports; the latter may be as varied as concrete, cement, plaster, wood, glass or metal, in the context of a paint, paper or card in the context of a paper coating, or skin in the context of a cosmetic formulation, although this list is not exhaustive.

The Applicant recalls that, in the case of mass fillers, the said fillers are added to the fibres before the sheet of paper is manufactured; one speaks of fillers added to the mass, or mass fillers, during the process of manufacture of the fibrous suspension feeding the paper machine. The said fillers can be added to the fibres in the form of suspensions and/or water-based dispersions of mineral matter. In the case of paper coatings, the latter are formulated from suspensions and/or water-based dispersions of mineral matter, and are applied on to supports (paper, plastic, card, etc.): one speaks in this case of coated papers when these supports are papers.

Finally, the Applicant designates under the term “dry powders”, products resulting from the drying of the dispersions and/or water-based suspensions of mineral matter defined above, which contain:
at least one mineral matter,
and at least one polymer, use of which is made in the invention.

In the context of this invention, the said dry powders are then used in the manufacture of plastics, with improved brightness and/or opacity. The said dry powders also enable water-based formulations of mineral matter to be manufactured, and notably paper coatings, mass fillers, water-based paints and cosmetic formulations. The said formulations then allow, after drying, dry products to be obtained, the opacity and/or brightness of which is improved. These dry products are papers filled by the previous mass fillers, or dry coverings which are dry films resulting from the drying of a paper coating, dry films resulting from the drying of a water-based paint formulation, or dry films resulting from the drying of a water-based cosmetic formulation.

In the present Application, the expression “dry products” thus designates both plastics and filled papers, but also “dry coverings”, i.e. dry coverings which are dry films resulting from the drying of a paper coating, dry films resulting from the drying of a water-based paint formulation, or again dry films resulting from the drying of a water-based cosmetic formulation.

In the context of the manufacture of water-based formulations containing mineral matter, such as, notably, paper coatings or water-based paints, the skilled man in the art keeps in mind the need to obtain an end product with improved optical properties, notably in terms of brightness, but also of opacity.

It is sought to attain this goal through the use of various additives, which may be included in the manufacture of the water-based dispersions and/or water-based suspensions of mineral matter (the latter are then used in the manufacture of the said water-based formulations), or which may be put directly in the said water-based formulations (the latter being obtained by direct mixing of its various constituents in water).

Independently of these two embodiments, the Applicant will now present the different means described in the prior art with a view to improving the optical properties of water-based formulations containing mineral matter, distinguishing between the case of solutions seeking to increase the brightness, those which concern the improvement of opacity, and finally those which allow both these properties to be improved simultaneously.

Historically, with a view to improving the brightness of water-based formulations of mineral matter, it is firstly known to use certain chemical structures, and notably those with an acrylic and/or methacrylic compound base, in order to synthesise polymers or copolymers which are introduced into the said water-based formulations of mineral fillers.

Thus, document JP 06 211 951 describes a copolymer produced from an alkyl methacrylate grouping having 1 to 4 carbon atoms, an alkyl methacrylate grouping having 6 to 30 carbon atoms, and a polyoxyalkyene grouping having a polymerisable function, used for the dispersion of pigments. As such, the said documents teaches that a paint manufactured from this copolymer and containing titanium dioxide has increased brightness.

Simultaneously, document U.S. Pat. No. 5,491,209 describes a copolymer with at least one ethylenically unsaturated monomer of polyethylene glycol, at least one monovinylic and aromatic monomer, and at least one monomer of the methacrylate type. This copolymer may be used in the formulation of paper coatings containing calcium carbonate, which enable sheets of paper with improved brightness to be obtained.

Finally, more recently, the skilled man in the art is also familiar with document WO 2004/041883, which describes the use of a hydrosoluble copolymer, preferably a weakly ionic one, which has at least one alkoxy or hydroxy polyalkylene glycol function grafted onto at least one ethylenic unsaturated monomer, as an agent improving the brightness of the end product, and notably brightness in the sheet of paper, whatever the angle of vision, i.e. an angle of between 20° and 85°, and more specifically between 45° and 75°.

Nevertheless, in addition to the chemical structure of these polymers, it proves to be the case that it is the technique of synthesis of such compounds which is most important with a view to obtaining water-based formulations of mineral fillers enabling dry coverings with improved brightness to be manufactured. More specifically, several patents (listed below) rapidly guide the skilled man in the art to the choice of “controlled structure” polymers. In the remainder of the present application the expression “process to obtain a polymer with a controlled structure or architecture” designates a process enabling the skilled man in the art to obtain a particular structure for the polymer which he wishes to produce (such as a block, comb, alternate, star-shape, statistic, etc.). Continuing his research in this field, the skilled man in the art thus familiarises himself with the document presenting the synthesis of controlled structure polymers, obtained by the ATRP (Atom Transfer Radical Polymerization) process. This technique has been amply described in the document (Controlled Radical Polymerization, K. Matyjaszewski, Am. Chem. Soc., 1998, Chap 16, pp 258).

Thus, document US 2003 0166755 describes polymers of the comb type with an acrylate monomer base, prepared using the ATRP process, which are effective dispersing agents for mineral fillers in water, such as calcium carbonate. In addition, the brightness (measured at 60°) of the dry coverings obtain using water-based formulations containing them is improved thereby (tables 2, 4, 5 and 6).

Similarly, document US 2004 0143032 describes compositions containing organic or inorganic pigments and block polymers obtained using the ATRP technique. The latter are effective dispersing agents of mineral matter in the aqueous phase, such as calcium carbonate. In addition, the water-based dispersions of mineral matter thus obtained can be used in the manufacture of inks, cosmetics or papers, in which they improve the brightness.

Finally, document US 2005 0004317 teaches a composition containing organic or inorganic pigments, and a mixture of a bloc copolymer by the ATRP technique and a compound of the organic acid or alkyl halide or sulphonic ester type, the said compound being likely to form a salt in water. Calcium carbonate is mentioned as a mineral filler, and the said mixtures of the copolymer and of the salt act as effective dispersing agents of the mineral filler in water, and enable the brightness of the end product to be improved.

Nevertheless, all these documents cause new problems to be revealed. Firstly, the ATRF process uses copper salt-based catalysts which caused undesirable pollution; the presence of these catalysts is indicated for the 3 previous documents, respectively in paragraphs 110, 110 and 118. Secondly, copper will also be found in the synthesised products, which the skilled man in the art does not necessarily desire. In addition, the ATRP process also uses amines, which are often undesirable in the end product.

With regard to this serious disadvantage, the skilled man in the art then turns to another polymerisation process enabling controlled structures to be obtained: the RAFT (Reversible Addition Fragmentation Transfer) technique. The latter has been amply described in the document (Controlled/Living Radical Polymerization-Progress in ATRP, NMP, and RAFT, K. Matyjaszewski, Am. Chem. Soc., 2000, Chap 20, pp 278).

The skilled man in the art then familiarises himself with document US 2004 0143035, which describes the use as dispersing agents of mineral fillers of grafted copolymers obtained by a process of controlled radical polymerisation, such as notably the RAFT technique, by bringing into contact monomers containing either hydrophilic groupings or hydrophobic groupings. The water-based dispersions thus obtained are used for the manufacture of various water-based formulations, which lead to paint films with improved brightness.

But a new problem, inherent to this polymerisation technique, then appears: this is the use of sulphurated transfer agents; as such, the previous document mentions the use of xanthogenates and thiocarboxylic esters (paragraph 089). Apart from the disadvantage that they are dangerous for the environment, these transfer agents give the polymers obtained a very unpleasant odour, and introduce into the end product organo-sulphurated molecules which are not necessarily desired.

To circumvent this new disadvantage, the skilled man in the art finally turns to recent controlled radical polymerisation techniques, which use nitroxides or alcoxyamines as polymerisation initiators.

Thus, document WO 00/71501 teaches him that particular polyalkoxyamines may be used to synthesise notably three-unit copolymers, in which each unit is derived from monomers as different as alkyl acrylates and styrenic derivatives, with excellent control of polymerisation and of the polydispersity index. However, this document does not reveal any particular use of the said polymers.

Finally, document WO 01/02345 teaches the skilled man in the art that polyalkoxyamines enable controlled structure polymers to be obtained, such as a block, comb, grafted shape, or statistic. These polymers have many applications as agents to modify rheology or dispersing agents of mineral fillers in the aqueous phase; however, this document contains no teaching concerning the use of such polymers to improve the optical properties of dry products obtained from water-based formulations of mineral matter containing the said polymers.

The state of the current technique, relative to technical solutions enabling the opacity of dry products obtained from the drying of water-based formulations of mineral matter to be improved is very different from the one previously described.

With this regard, it is known to manufacture calcium carbonate powders with a uniform granulometry, notably between 0.3±0.1 μm, as taught in document U.S. Pat. No. 5,910,214. Coated papers derived from such products have improved opacity and whiteness.

It is also known to increase the dry matter concentration in paper coatings, in order to increase the opacity of the end product, as described in document WO 02/48459 (page 4, 11 28-29). The means to accomplish this consists here in the use of a branched amide polyester in the said coatings.

According to document EP 1 347 834, it is also known to grind water-based suspensions of calcium carbonate with specific grinding aid agents, such as acrylic acid and methacrylic acid copolymers which have been partially neutralised by magnesium, barium, zinc, aluminium and/or diamine ions. Manufactured papers incorporating such mineral fillers thus refined that have a high opacity.

Another well-known means to increase the opacity of papers is the use of organic, spherical and hollow pigments, as described in document EP 0 959 176. Paper coatings containing the said pigments then enable the opacity of the end product to be improved. The prior art also mentions documents which describe technical solutions enabling the brightness and the opacity to be improved jointly.

This is the case of document WO/03 08511, which describes a suspension of titanium dioxide of the rutile type, also containing a dispersing agent and an amine, the use of which in water-based paints enables not only the opacity of the end product, but also its brightness, to be improved (page 21, 11 20-22).

Document WO 2004/059079 proposes another solution to this end, which consists in mixing a natural calcium carbonate having a distribution factor of between 30 and 45, and a precipitate calcium carbonate having a distribution factor of between 55 and 75. Coatings formulated from water-based suspensions of this mixture of pigments enable sheets of paper to be manufactured, the opacity, brightness and whiteness of which are improved.

Document EP 1 230 160 describes a process for manufacturing a synthetic calcium carbonate, enabling the granulometric distribution of the particles obtained to be controlled precisely. Papers containing these mineral fillers then have improved opacity, brightness and whiteness.

Finally, document JP 57 106 797 describes a water-based suspension of calcium carbonate treated by a copolymer of acrylic acid, with maleic or sulphonic or lignosulphonic or phosphoric acid. Coatings manufactured from water-based suspensions of these treated fillers enable sheets of paper to be manufactured, the opacity, brightness and whiteness of which are improved.

Examining the solutions proposed by the documents relative to a specific increase of brightness, the skilled man in the art draws the lesson that, in addition to the chemical nature of the agents used to this end, the fact of obtaining polymers with a controlled architecture plays an important role. With this regard, the techniques of polymerisation described in the prior art prove to be unsuitable, in the sense that they pose pollution problems relating to the use of polymers manufactured from copper-based compounds, or odour problems of polymers obtained by the use of sulphur-based compounds, or again pollution problems which may be represented by the incorporation of organo-sulphurated molecules.

Examining the state of the technique relative to a specific increase of opacity, the skilled man in the art learns of various solutions, such as the use of particular acrylic polymers, the manufacture of a suspension of mineral filler having a uniform granulometry, an increase of the content by weight of mineral matter in a paper coating or, again, incorporation in the said coatings of organic, spherical and hollow pigments. However, nothing teaches the skilled man in the art the use of controlled architecture polymers, with a view to increasing the opacity of dry coverings containing mineral fillers.

Finally, examining the state of the technique relative to a joint increase of brightness and opacity, the skilled man in the art learns of different means, such as the mixing of two calcium carbonates of a particular distribution of sizes, the manufacture of a precipitated calcium carbonate having a very specific granulometry or, again, treatment by acrylic copolymers of a water-based suspension of calcium carbonate. As previously, nothing reveals in these documents the use of controlled architecture polymers, with a view to increasing the opacity and brightness of dry coverings containing mineral fillers. In addition, all these solutions are based on the manufacture of a suspension of mineral fillers, which suspension is then used in the composition of a paper coating. This approach gives the paper manufacturer only a slight flexibility, and they must content themselves with using the said suspensions as such: they have no direct means to improve the brightness of the manufactured papers, such as a possible additive, the dosage of which they could control in their paper coating.

Continuing his research, the Applicant has found, in a surprising manner, the solution to the problem of obtaining water-based formulations of mineral matter, enabling the brightness or opacity, or jointly both these properties, to be improved in the final product, through the use of controlled architecture hydrosoluble polymers, obtained through the use of a controlled radical polymerisation process using a particular alcoxyamine.

The said process has neither the pollution problems relating to the use of polymers manufactured from copper-based compounds, nor the odour problems of polymers obtained through the use of sulphur-based compounds, nor the pollution problems which the incorporation of organo-sulphurated molecules may represent. In a very surprising manner, the use of controlled architecture polymers obtained by the said process leads to an increase of brightness or opacity, or to a joint increase of both these properties, in dry mineral matter-based products containing the said polymers. These various possibilities leads to a greater flexibility offered to the end compounder, who may thus choose to increase the property of their choice (or both of them), depending on their schedule of specifications.

A first object of the invention is thus the use in a manufacturing process of mineral matter-based dry products, as agents improving opacity and/or brightness of the said dry products, of hydrosoluble polymers of controlled structure obtained by a process of controlled radical polymerisation using, as the polymerisation initiator, a particular alcoxyamine of general formula (A). In a first variant, this use of the said hydrosoluble polymers is characterised in that the manufacturing process of the said dry products includes at least one stage of grinding and/or at least one stage of dispersion and/or at least one stage of concentration in water of the mineral matter, which leads to a dispersion and/or aqueous suspension of mineral matter being obtained.

The dispersion and/or aqueous suspension of the mineral matter thus obtained enables the manufacture of water-based formulations of mineral matter, in the fields of paper and notably in paper coatings and in mass filler, in water-based paints, and in cosmetic compositions.

The said formulations then allow, after drying, dry products to be obtained the opacity and/or brightness of which is improved, dry products which are either papers filled using the previous mass fillers, or dry coverings which are dry films resulting from the drying of the said paper coatings, or dry paint films resulting from the drying of the said water-based paint formulations or, finally, dry films resulting from the drying of the said water-based cosmetic formulations.

Moreover, the dispersion and/or water-based suspension of mineral matter previously obtained may be dried; the invention then concerns also the dry powders thus obtained. The said dry powders then allow the manufacture of plastics the opacity and/or brightness of which is improved. The said dry powders also enable water-based formulations of mineral matter to be manufactured, and notably paper coatings, mass fillers, water-based paints and cosmetic formulations. These said formulations then allow, after drying, dry products to be obtained the opacity and/or brightness of which is improved, dry products which are papers filled using the previous mass fillers, or dry coverings which are dry films resulting from the drying of the said paper coatings, or dry paint films resulting from the drying of the said water-based paint formulations, or, finally, dry films resulting from the drying of the said water-based cosmetic formulations.

In a second variant, the invention consists in that the use in a process of manufacture of mineral matter-based dry products is characterised in that the said polymers are used as direct additives in the field of plastics, and in the field of water-based formulations, and notably in the paper sectors, and particularly in paper coatings, in water-based paints and in cosmetic compositions.

The said formulations then allow, after drying, dry products to be obtained the opacity and/or brightness of which is improved, which are dry coverings, i.e. dry films resulting from the drying of the said paper coatings, dry paint films resulting from the drying of the said water-based paint formulations, and dry films resulting from the drying of the said water-based cosmetic formulations, while the direct use according to the invention of the said polymers in plastics leads to plastics being obtained the opacity and/or brightness of which is improved.

Secondly, the invention concerns the use of a dispersion and/or water-based suspension of mineral matter containing the said polymers, in a process for manufacturing dry products containing mineral matter.

The dispersion and/or aqueous suspension of the mineral matter thus obtained enables the manufacture of water-based formulations of mineral matter, in the fields of paper and notably in paper coatings and in mass filler, in water-based paints, and in cosmetic compositions.

The said formulations then allow, after drying, dry products to be obtained the opacity and/or brightness of which is improved, dry products which are papers filled using the said mass fillers, or dry coverings and notably dry films resulting from the drying of the said paper coatings, or dry paint films resulting from the drying of the said water-based paint formulations or, finally, dry films resulting from the drying of the said water-based cosmetic formulations.

Moreover, the dispersion and/or water-based suspension of mineral matter previously obtained may be dried, and the dry powders thus obtained then allow the manufacture of plastics, the opacity and/or brightness of which is improved. The said dry powders also enable water-based formulations of mineral matter to be manufactured, and notably paper coatings, mass fillers, water-based paints and cosmetic formulations. These said formulations then allow, after drying, dry products to be obtained the opacity and/or brightness of which is improved, dry products which are papers filled using the previous mass fillers, or dry coverings which are dry films resulting from the drying of the said paper coatings, or dry paint films resulting from the drying of the said water-based paint formulations, or, finally, dry films resulting from the drying of the said water-based cosmetic formulations.

The Applicant has thus found, in a surprising manner, that hydrosoluble polymers with a controlled structure obtained by a process of controlled radical polymerisation using an alcoxyamine of a particular formula (A), can be used very effectively in a process to manufacture dry products containing mineral matter, as an agent improving opacity and/or brightness of the said products.

This polymerisation process uses, as an initiator of polymerisation, a particular alcoxyamine of general formula (A):

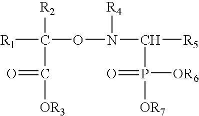

where:
R₁ and R₂ represent an alkyl radical, linear or branched, with a number of carbon atoms ranging from 1 to 5,
R₃ represents a hydrogen atom, an alkyl radical, linear or branched, with a number of carbon atoms ranging from 1 to 8, a phenyl radical, a cation such as L+, Na+, K+, H4N+, Bu3HN⁺, where Bu=butyl,
R₄ represents an alkyl radical, linear or branched, with a number of carbon atoms ranging from 1 to 8, and preferentially a tertiobutyl radical,
R₅ represents an alkyl radical, linear or branched, with a number of carbon atoms ranging from 1 to 8, and preferentially a tertiobutyl radical,
R₆ and R₇ represent an alkyl radical, linear or branched, with a number of carbon atoms ranging from 1 to 8, and preferentially an ethyl radical,

The polymers than obtained by the said process have the advantage that they do not contain any copper salt-based compounds, unlike polymers obtained by ATRP, or sulphurated compounds, unlike products obtained using the RAFT technique, whilst having an architecture which may be controlled through their polymerisation process.

The Applicant stresses that it is familiar with the patent application which has not yet been published, FR 03 15385, which describes the use as dispersing agents and agents to aid grinding of mineral matter, of controlled structure hydrosoluble polymers obtained by the process of controlled radical polymerisation using an alcoxyamine of particular formula (A). This document does not describe the use of such polymers as agents improving the optical properties of the water-based formulations of mineral matter containing it. Nor does it describe the influence of such polymers on the optical properties of the water-based formulations of mineral matter containing them.

The first object of the invention is thus the use in a manufacturing process of dry products containing mineral matter, as agents improving opacity and/or brightness of the said products, of hydrosoluble polymers of controlled structure obtained by a process of controlled radical polymerisation using, as the polymerisation initiator, a particular alcoxyamine of general formula (A):

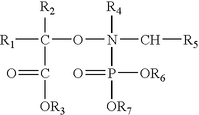

where:
R₁ and R₂ represent an alkyl radical, linear or branched, with a number of carbon atoms ranging from 1 to 5,
R₃ represents a hydrogen atom, an alkyl radical, linear or branched, with a number of carbon atoms ranging from 1 to 8, a phenyl radical, a cation such as L+, Na+, K+, H4N+, Bu3HN⁺, where Bu=butyl,
R₄ represents an alkyl radical, linear or branched, with a number of carbon atoms ranging from 1 to 8, and preferentially a tertiobutyl radical,
R₅ represents an alkyl radical, linear or branched, with a number of carbon atoms ranging from 1 to 8, and preferentially a tertiobutyl radical,
R₆ and R₇ represent an alkyl radical, linear or branched, with a number of carbon atoms ranging from 1 to 8, and preferentially an ethyl radical,

The invention thus enables dry products containing mineral matter with improved optical properties, such as notably their brightness and/or their opacity, to be obtained.

These aims are attained thanks to the use of hydrosoluble polymers in a process to manufacture dry products containing mineral matter, which is characterised in that the said polymers have a controlled structure and are obtained by a process of controlled radical polymerisation using as an initiator of polymerisation a particular alcoxyamine of general formula (A).

In a first variant, this use of the said hydrosoluble polymers is characterised in that the manufacturing process of the said dry products includes at least one stage of grinding and/or at least one stage of dispersion and/or at least one stage of concentration in water of the mineral matter, which leads to a dispersion and/or aqueous suspension of mineral matter being obtained.

The dispersion and/or aqueous suspension of the mineral matter thus obtained enables the manufacture of water-based formulations of mineral matter, in the fields of paper and notably in paper coatings and in mass filler, in water-based paints, and in cosmetic compositions.

The said formulations then allow the manufacture of dry coverings the opacity and/or the brightness of which is improved, in the paper fields, and notably in papers coated using the previous paper coatings and in papers manufactured from the previous mass fillers, in paint films, and in films obtained from cosmetic compositions.

Moreover, the dispersion and/or water-based suspension of mineral matter previously obtained may be dried; the invention then concerns also the dry powders thus obtained. The said dry powders then allow the manufacture of plastics the opacity and/or brightness of which is improved. The said dry powders also enable water-based formulations of mineral matter to be manufactured, and notably paper coatings, mass fillers, water-based paints and cosmetic formulations. These said formulations enable dry products to be manufactured which are dry coverings the opacity and/or brightness of which is improved, and notably papers coated using the said paper coatings and papers manufactured from the said mass fillers, paint films and films obtained from the said cosmetic formulations.

In a second variant, the invention consists in that the use in a process of manufacture of mineral matter-based dry products is characterised in that the said polymers are used as direct additives in water-based formulations, and notably in the paper fields, and particularly in paper coatings, in plastics, in water-based paints and in cosmetic compositions, and also in the field of plastics.

The said formulations then allow the manufacture of dry coverings the opacity and/or the brightness of which is improved, in the fields of paper, and notably in coated papers, in paint films, in films obtained from cosmetic compositions, while the direct use according to the invention of the said polymers in plastics leads to plastics being obtained the opacity and/or brightness of which is improved.

The use according to the invention of hydrosoluble polymers is also characterised in that the polymers are obtained by a process of controlled radical polymerisation, using the previously described particular alcoxyamine, of monomers chosen from:
a) at least one ionic monomer which is either:
i) anionic with a carboxylic or dicarboxylic or phosphoric or phosphonic or sulphonic function, or their mixtures, or
ii) cationic, or
iii) a mixture of i) and ii)
b) and at least one non-ionic monomer, where the non-ionic monomer consists of at least one monomer of formula (I):

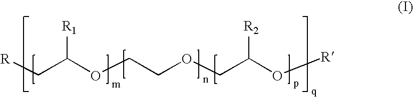

where:
m and p represent a number of alkylene oxide units of less than or equal to 150,
n represents a number of ethylene oxide units of less than or equal to 150,
q represents a whole number at least equal to 1 and such that 5≦(m+n+p)q≦150, and preferentially such that 15≦(m+n+p)q≦120,
R₁ represents hydrogen or the methyl or ethyl radical,
R₂ represents hydrogen or the methyl or ethyl radical,
R represents a radical containing an unsaturated polymerisable function, belonging to the group of vinylics, or to the group of acrylic, methacrylic, maleic, itaconic, crotonic, vinylphthalic esters, or to the group of unsaturated urethanes such as, for example, acrylurethane, methacrylurethane, α-α′ dimethyl-isopropenyl-benzylurethane, allylurethane, or to the group of allylic or vinylic ethers, whether or not substituted, or again to the group of ethylenically unsaturated amides or imides,
R′ represents hydrogen or a hydrocarbonate radical having 1 to 40 atoms of carbon, and represents preferentially a hydrocarbonate radical having 1 to 12 carbon atoms, and very preferentially a hydrocarbonate radical having 1 to 4 carbon atoms,
   or a mixture of several monomers of formula (I),
c) and possibly at least one monomer of the acrylamide or methacrylamide type and their mixtures, or again at least one non-hydrosoluble monomer such as the alkyl acrylates or methacrylates, the vinylics such as vinyl acetate, vinylpyrrolidone, styrene, alphamethylstyrene and their derivatives, or again at least one organofluorate or organosililate monomer, or their mixtures,
d) and possibly at least one monomer having at least two ethylenic unsaturations called in the remainder of the application a crosslinking monomer, or a mixture of several of these monomers.

In a particular manner, the use according to the invention of hydrosoluble polymers is also characterised in that the hydrosoluble polymers are obtained by controlled radical polymerisation of monomers chosen from among:
a) at least one ionic monomer which is either:
i) anionic with ethylenic unsaturation and with a monocarboxylic function in the acid or acid-salt state chosen from among the ethylenic unsaturation monomers and with a monocarboxylic function such as acrylic or methacrylic acid, or again the diacid hemiesters such as the C₁ to C₄ monoesters of maleic or itaconic acids, or chosen from among the monomers with ethylenic unsaturation and with a dicarboxylic function in the acid or acid-salt state such as crotonic, isocrotonic, cinnamic, itaconic, maleic acid, or again the anhydrides of carboxylic acids, such as maleic anhydride, or chosen from among the monomers with ethylenic unsaturation and with a sulphonic function in the acid or acid-salt state such as 2-acrylamido-2-methyl-propane-sulphonic acid, sodium methallylsulphonate, vinyl sulphonic acid and styrene sulphonic acid, or again chosen from among the monomers with ethylenic unsaturation and with a phosphoric function in the acid or acid-salt state such as vinyl phosphoric acid, ethylene glycol methacrylate phosphate, propylene glycol methacrylate phosphate, ethylene glycol acrylate phosphate, propylene glycol acrylate phosphate and their ethoxylates, or again chosen from among the monomers with ethylenic unsaturation and with a phosphonic function in the acid or acid-salt state, such as vinyl phosphonic acid, or their mixtures, or
ii) cationic chosen from among N-[3-(dimethylamino) propyl]acrylamide or N-[3-(dimethylamino) propyl]methacrylamide, the unsaturated esters such as N-[2-(dimethylamino) ethyl]methacrylate, or N-[2-(dimethylamino) ethyl]acrylate, or from among the quaternary ammonium compounds such as [2-(methacryloyloxy)ethyl]trimethyl ammonium chloride or sulphate, [2-(acryloyloxy)ethyl]trimethyl ammonium chloride or sulphate, [3-(acrylamido) propyl]trimethyl ammonium chloride or sulphate, dimethyl diallyl ammonium chloride or sulphate, [3-(methacrylamido) propyl]trimethyl ammonium chloride or sulphate, or their mixtures, or
iii) a mixture of the abovementioned anionic and cationic monomers
b) and a least one monomer with a non-ionic ethylenic unsaturation of formula (I):

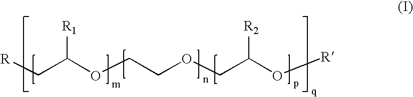

where:
m and p represent a number of alkylene oxide units of less than or equal to 150,
n represents a number of ethylene oxide units of less than or equal to 150,
q represents a whole number at least equal to 1 and such that 5≦(m+n+p)q ≦150, and preferentially such that 15≦(m+n+p)q≦120,
R₁ represents hydrogen or the methyl or ethyl radical,
R₂ represents hydrogen or the methyl or ethyl radical,
R represents a radical containing an unsaturated polymerisable function, belonging to the group of vinylics, or to the group of acrylic, methacrylic, maleic, itaconic, crotonic, vinylphthalic esters, or to the group of unsaturated urethanes such as, for example, acrylurethane, methacrylurethane, α-α′ dimethyl-isopropenyl-benzylurethane, allylurethane, or to the group of allylic or vinylic ethers, whether or not substituted, or again to the group of ethylenically unsaturated amides or imides,
R′ represents hydrogen or a hydrocarbonate radical having 1 to 40 atoms of carbon, and represents preferentially a hydrocarbonate radical having 1 to 12 carbon atoms, and very preferentially a hydrocarbonate radical having 1 to 4 carbon atoms,

or a mixture of several monomers of formula (I),
c) and possibly at least one monomer of the acrylamide or methacrylamide type and their mixtures, or again at least one non-hydrosoluble monomer such as the alkyl acrylates or methacrylates, the vinylics such as vinyl acetate, vinylpyrrolidone, styrene, alphamethylstyrene and their derivatives, or again at least one organofluorate or organosililate monomer chosen preferentially from among the molecules of formulae (IIa) or (IIb):

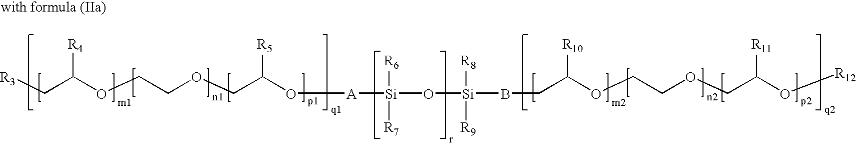

where:
m₁, p₁, m₂ and p₂ represent a number of alkylene oxide units of less than or equal to 150,
n₁ et n₂ represent a number of ethylene oxide units of less than or equal to 150,
q₁ and q₂ represent a whole number at least equal to 1 and such that 0≦(m₁+n₁+p₁)q₁≦150 and 0≦(m₂+n₂+p₂)q₂≦150,
r represents a number such that 1≦r≦200,
R₃ represents a radical containing an unsaturated polymerisable function, belonging to the group of vinylics, or to the group of acrylic, methacrylic, maleic, itaconic, crotonic, vinylphthalic esters, or to the group of unsaturated urethanes such as, for example, acrylurethane, methacrylurethane, α-α′ dimethyl-isopropenyl-benzylurethane, allylurethane, or to the group of allylic or vinylic ethers, whether or not substituted, or again to the group of ethylenically unsaturated amides or imides,
R₄, R₅, R₁₀ et R₁₁, represent hydrogen or the methyl or ethyl radical,
R₆, R₇, R₈ and R₉ represent linear or branched alkyl or aryl, or alkylaryl or arylalkyl groupings, having 1 to 20 carbon atoms, or their mixtures,
R₁₂ represents a hydrocarbonated radical having 1 to 40 carbon atoms,
A and B are groupings which may be present, which then represent a hydrocarbonated radical having 1 to 4 carbon atoms,
   with formula (IIb)

R-A-Si(OB)₃

where:
R represents a radical containing an unsaturated polymerisable function, belonging to the group of vinylics, or to the group of acrylic, methacrylic, maleic, itaconic, crotonic, vinylphthalic esters, or to the group of unsaturated urethanes such as, for example, acrylurethane, methacrylurethane, α-α′ dimethyl-isopropenyl-benzylurethane, allylurethane, or to the group of allylic or vinylic ethers, whether or not substituted, or again to the group of ethylenically unsaturated amides or imides,
A is a grouping which may be present, which then represents a hydrocarbonated radical having 1 to 4 carbon atoms,
B represents a hydrocarbonated radical having 1 to 4 carbon atoms, or the mixture of several of these monomers,
d) and possibly at least one crosslinking monomer chosen from the group constituted by ethylene glycol dimethacrylate, trimethylolpropanetriacrylate, allyl acrylate, the allyl maleates, methylene-bis-acrylamide, methylene-bis-methacrylamide, tetrallyloxyethane, the triallylcyanurates, the allyl ethers obtained from polyols such as pentaerythritol, sorbitol, sucrose or others, or chosen from among the molecules of formula (III):

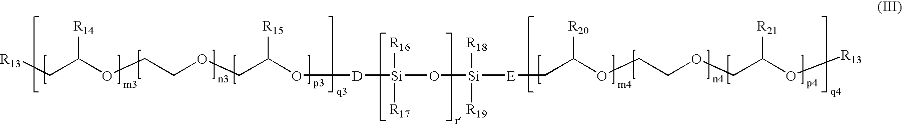

where:
m₃, p₃, m₄ and p₄ represent a number of alkylene oxide units of less than or equal to 150,
n₃ et n₄ represent a number of ethylene oxide units of less than or equal to 150,
q₃ and q₄ represent a whole number at least equal to 1 and such that 0≦(m₃+n₃+p₃)q₃≦150 and 0≦(m₄+n₄+p₄)q₄≦150,
r′ represents a number such that 1≦r′≦200,
R₁₃ represents a radical containing an unsaturated polymerisable function, belonging to the group of vinylics, or to the group of acrylic, methacrylic, maleic, itaconic, crotonic, vinylphthalic esters, or to the group of unsaturated urethanes such as, for example, acrylurethane, methacrylurethane, α-α′ dimethyl-isopropenyl-benzylurethane, allylurethane, or to the group of allylic or vinylic ethers, whether or not substituted, or again to the group of ethylenically unsaturated amides or imides,
R₁₄, R₁₅, R₂₀ et R₂₁ represent hydrogen or the methyl or ethyl radical,
R₁₆, R₁₇, R₁₈ and R₁₉ represent linear or branched alkyl or aryl, or alkylaryl or arylalkyl groupings, having 1 to 20 carbon atoms, or their mixtures,
D and E are groupings which may be present, which then represent a hydrocarbonated radical having 1 to 4 carbon atoms,

or the mixture of several of these monomers.

More specifically, the use according to the invention of hydrosoluble polymers is characterised in that the said abovementioned polymers are constituted, expressed in weight, relative to the total weight of the monomers used:
a) from 2% to 98% and yet more particularly from 5% to 95% of a least one ionic monomer which is either:
i) an anionic monomer with ethylenic unsaturation and with a monocarboxylic function in the acid or acid-salt state chosen from among the ethylenic unsaturation monomers and with a monocarboxylic function such as acrylic or methacrylic acid, or again the diacid hemiesters such as the C₁ to C₄ monoesters of maleic or itaconic acids, or chosen from among the monomers with ethylenic unsaturation and with a dicarboxylic function in the acid or acid-salt state such as crotonic, isocrotonic, cinnamic, itaconic, maleic acid, or again the anhydrides of carboxylic acids, such as maleic anhydride, or chosen from among the monomers with ethylenic unsaturation and with a sulphonic function in the acid or acid-salt state such as 2-acrylamido-2-methyl-propane-sulphonic acid, sodium methallylsulphonate, vinyl sulphonic acid and styrene sulphonic acid, or again chosen from among the monomers with ethylenic unsaturation and with a phosphoric function in the acid or acid-salt state such as vinyl phosphoric acid, ethylene glycol methacrylate phosphate, propylene glycol methacrylate phosphate, ethylene glycol acrylate phosphate, propylene glycol acrylate phosphate and their ethoxylates, or again chosen from among the monomers with ethylenic unsaturation and with a phosphonic function in the acid or acid-salt state, such as vinyl phosphonic acid, or their mixtures, or
ii) a cationic monomer chosen from among N-[3-(dimethylamino) propyl]acrylamide or N-[3-(dimethylamino) propyl]methacrylamide, the unsaturated esters such as N-[2-(dimethylamino) ethyl]methacrylate, or N-[2-(dimethylamino) ethyl]acrylate, or from among the quaternary ammonium compounds such as [2-(methacryloyloxy)ethyl]trimethyl ammonium chloride or sulphate, [2-(acryloyloxy)ethyl]trimethyl ammonium chloride or sulphate, [3-(acrylamido) propyl]trimethyl ammonium chloride or sulphate, dimethyl diallyl ammonium chloride or sulphate, [3-(methacrylamido) propyl]trimethyl ammonium chloride or sulphate, or their mixtures, or
iii) a mixture of the abovementioned anionic and cationic monomers,
b) from 2 to 98% and yet more particularly from 5% to 95% of a least one monomer with non-ionic ethylenic unsaturation of formula (I):

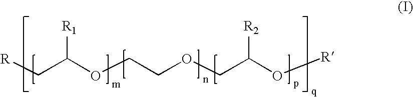

where:
m and p represent a number of alkylene oxide units of less than or equal to 150,
n represents a number of ethylene oxide units of less than or equal to 150,
q represents a whole number at least equal to 1 and such that 5≦(m+n+p)q ≦150, and preferentially such that 15≦(m+n+p)q≦120,
R₁ represents hydrogen or the methyl or ethyl radical,
R₂ represents hydrogen or the methyl or ethyl radical,
R represents a radical containing an unsaturated polymerisable function, belonging to the group of vinylics, or to the group of acrylic, methacrylic, maleic, itaconic, crotonic, vinylphthalic esters, or to the group of unsaturated urethanes such as, for example, acrylurethane, methacrylurethane, α-α′ dimethyl-isopropenyl-benzylurethane, allylurethane, or to the group of allylic or vinylic ethers, whether or not substituted, or again to the group of ethylenically unsaturated amides or imides,
R′ represents hydrogen or a hydrocarbonate radical having 1 to 40 atoms of carbon, and represents preferentially a hydrocarbonate radical having 1 to 12 carbon atoms, and very preferentially a hydrocarbonate radical having 1 to 4 carbon atoms,

or a mixture of several monomers of formula (I),
c) from 0 to 50% of at least one monomer of the acrylamide or methacrylamide type and their mixtures, or again at least one non-hydrosoluble monomer such as the alkyl acrylates or methacrylates, the vinylics such as vinyl acetate, vinylpyrrolidone, styrene, alphamethylstyrene and their derivatives, or again at least one organofluorate or organosililate monomer chosen preferentially from among the molecules of formulae (IIa) or (IIb):

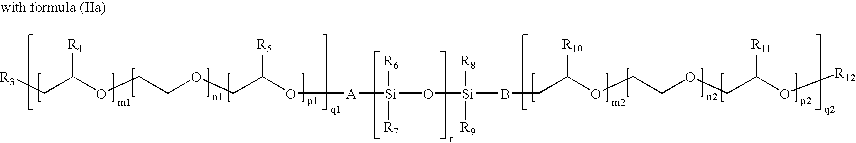

where:
m₁, p₁, m₂ and p₂ represent a number of alkylene oxide units of less than or equal to 150,
n₁ et n₂ represent a number of ethylene oxide units of less than or equal to 150,
q₁ and q₂ represent a whole number at least equal to 1 and such that 0≦(m₁+n₁+p₁)q₁≦150 and 0≦(m₂+n₂+p₂)q₂≦150,
r represents a number such that 1≦r≦200,
R₃ represents a radical containing an unsaturated polymerisable function, belonging to the group of vinylics, or to the group of acrylic, methacrylic, maleic, itaconic, crotonic, vinylphthalic esters, or to the group of unsaturated urethanes such as, for example, acrylurethane, methacrylurethane, α-α′ dimethyl-isopropenyl-benzylurethane, allylurethane, or to the group of allylic or vinylic ethers, whether or not substituted, or again to the group of ethylenically unsaturated amides or imides,
R₄, R₅, R₁₀ et R₁₁ represent hydrogen or the methyl or ethyl radical,
R₆, R₇, R₈ and R₉ represent linear or branched alkyl or aryl, or alkylaryl or arylalkyl groupings, having 1 to 20 carbon atoms, or their mixtures,
R₁₂ represents a hydrocarbonated radical having 1 to 40 carbon atoms,
A and B are groupings which may be present, which then represent a hydrocarbonated radical having 1 to 4 carbon atoms,

with formula (IIb)

R-A-Si(OB)₃

where:
R represents a radical containing an unsaturated polymerisable function, belonging to the group of vinylics, or to the group of acrylic, methacrylic, maleic, itaconic, crotonic, vinylphthalic esters, or to the group of unsaturated urethanes such as, for example, acrylurethane, methacrylurethane, α-α′ dimethyl-isopropenyl-benzylurethane, allylurethane, or to the group of allylic or vinylic ethers, whether or not substituted, or again to the group of ethylenically unsaturated amides or imides,
A is a grouping which may be present, which then represents a hydrocarbonated radical having 1 to 4 carbon atoms,
B represents a hydrocarbonated radical having 1 to 4 carbon atoms, or the mixture of several of these monomers,
d) from 0 to 3% of at least one crosslinking monomer chosen from the group constituted by ethylene glycol dimethacrylate, trimethylolpropanetriacrylate, allyl acrylate, the allyl maleates, methylene-bis-acrylamide, methylene-bis-methacrylamide, tetrallyloxyethane, the triallylcyanurates, the allyl ethers obtained from polyols such as pentaerythritol, sorbitol, sucrose or others, or chosen from among the molecules of formula (III):

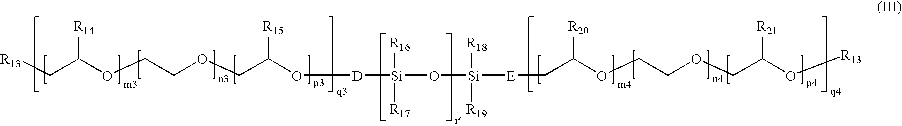

where:
m₃, p₃, m₄ and p₄ represent a number of alkylene oxide units of less than or equal to 150,
n₃ et nm represent a number of ethylene oxide units of less than or equal to 150,
q₃ and q₄ represent a whole number at least equal to 1 and such that 0≦(m₃+n₃+p₃)q₃≦150 and 0≦(m₄+n₄+p₄)q₄≦150,
r′ represents a number such that 1≦r′≦200,
R₁₃ represents a radical containing an unsaturated polymerisable function, belonging to the group of vinylics, or to the group of acrylic, methacrylic, maleic, itaconic, crotonic, vinylphthalic esters, or to the group of unsaturated urethanes such as, for example, acrylurethane, methacrylurethane, α-α′ dimethyl-isopropenyl-benzylurethane, allylurethane, or to the group of allylic or vinylic ethers, whether or not substituted, or again to the group of ethylenically unsaturated amides or imides,
R₁₄, R₁₅, R₂₀ et R₂₁ represent hydrogen or the methyl or ethyl radical,
R₁₆, R₁₇, R₁₈ and R₁₉ represent linear or branched alkyl or aryl, or alkylaryl or arylalkyl groupings, having 1 to 20 carbon atoms, or their mixtures,
D and E are groupings which may be present, which then represent a hydrocarbonated radical having 1 to 4 carbon atoms,

or the mixture of several of these monomers.

This use according to the invention of hydrosoluble polymers is also characterised in that the said polymers are obtained in the acid form and possibly distilled, and may be partially or totally neutralised by one or more agents such as the metal oxides or hydroxides, such as, for example, those of aluminium, and notably oxides or hydroxides of alkaline metals, such as, for example, sodium, potassium, lithium, or oxides or hydroxides of the alkaline earths such as, for example, calcium, magnesium, barium, or again the oxides or hydroxides of transition metals such as, for example, zinc, copper, iron, or again by ammonia or by the primary, secondary or tertiary aliphatic and/or cyclic amines such as, for example, stearylamine, the ethanolamines (mono-, di-, tri-ethanolamine), mono- and diethylamine, cyclohexamine, methylcyclohexylamine, amino methyl propanol, or morpholine.

According to another variant, the said polymers may also be before or after total or partial neutralisation reaction, treated and separated into several phases, according to static or dynamic processes known to the skilled man in the art, by one or more polar solvents belonging notably to the group constituted by water, methanol, ethanol, propanol, isopropanol, the butanols, acetone, tetrahydrofuran or their mixtures.

According to another variant, the said polymers may possibly be dried by one of the techniques well known to the skilled man in the art.

This use according to the invention of hydrosoluble polymers is also characterised in that the said polymers are hydrosoluble copolymers and have a structure of the statistic, block, comb, grafted or alternate type.

Depending on its use, the skilled man in the art will be able to adapt the molecular weight of the polymers used according to the invention.

This molecular weight is determined using the GPC (Gel Permeability Chromatography) method, using a liquid chromatography device of Waters™ brand fitted with two detectors, one of which combines dynamic diffusion of light with viscometry measured using a Viscotek™ viscometer, the other being a detector of refractometric concentration of brand Waters™.

This liquid chromatography equipment is fitted with steric exclusion columns suitably chosen by the skilled man in the art in order to separate the different molecular weights of the polymers studied.

The elution liquid phase is an aqueous phase.

This use is also characterised in that the mineral matter is chosen from among a pigment and/or a mineral filler, chosen from among natural or synthetic calcium carbonate, the dolomites, kaolin, talc, cement, gypsum, lime, magnesia, titanium dioxide, satin white, aluminium trioxide, or again aluminium trihydroxide, the silicas, mica and a mixture of these fillers one with another, such as talc-calcium carbonate mixtures, calcium carbonate-kaolin mixtures, or again mixtures of calcium carbonate with aluminium trihydroxide or aluminium trioxide, or again mixtures with synthetic or natural fibres, or again mineral costructures such as talc-calcium carbonate costructures or talc-titanium dioxide costructures, or their mixtures.

Preferentially, this use is characterised in that the said mineral matter is natural or synthetic calcium carbonate, and more specifically a natural calcium carbonate chosen from among marble, calcite, chalk or their mixtures.

Secondly, the invention concerns the use of a dispersion and/or water-based suspension of mineral matter containing the said polymers, in a process for manufacturing dry products containing mineral matter, as agents improving the opacity and/or brightness of the said dry products.

The dispersion and/or aqueous suspension of the mineral matter obtained enables the manufacture of water-based formulations of mineral matter, in the fields of paper and notably in paper coatings and in mass filler, in water-based paints, and in cosmetic compositions.

The said formulations then allow, after drying, dry products to be obtained the opacity and/or brightness of which is improved, dry products which are papers filled using the said mass fillers, or dry coverings which are dry films resulting from the drying of the said paper coatings, or dry films resulting from the drying of the said water-based paint formulations or, finally, dry films resulting from the drying of the said water-based cosmetic formulations.

Moreover, the dispersion and/or water-based suspension of mineral matter previously obtained may be dried, and the dry powders thus obtained then allow the manufacture of plastics, the opacity and/or brightness of which is improved. The said dry powders also enable water-based formulations of mineral matter to be manufactured, and notably paper coatings, mass fillers, water-based paints and cosmetic formulations. The said formulations then allow, after drying, dry products to be obtained the opacity and/or brightness of which is improved, dry products which are papers filled using the said mass fillers, or dry coverings which are dry films resulting from the drying of the said paper coatings, or dry films resulting from the drying of the said water-based paint formulations or, finally, dry films resulting from the drying of the said water-based cosmetic formulations.

The use according to the invention of a dispersion and/or of suspension containing the said hydrosoluble polymers is also characterised in that the said polymers are obtained by a process of controlled radical polymerisation, using the previously described particular alcoxyamine, of monomers chosen from:
a) at least one ionic monomer which is either:
i) anionic with a carboxylic or dicarboxylic or phosphoric or phosphonic or sulphonic function, or their mixtures, or
ii) cationic, or
iii) a mixture of i) and ii)
   b) and at least one non-ionic monomer, where the non-ionic monomer consists of at least one monomer of formula (J):

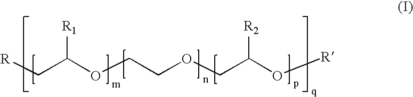

where:
m and p represent a number of alkylene oxide units of less than or equal to 150,
n represents a number of ethylene oxide units of less than or equal to 150,
q represents a whole number at least equal to 1 and such that 5≦(m+n+p)q≦150, and preferentially such that 15≦(m+n+p)q≦120,
R₁ represents hydrogen or the methyl or ethyl radical,
R₂ represents hydrogen or the methyl or ethyl radical,
R represents a radical containing an unsaturated polymerisable function, belonging to the group of vinylics, or to the group of acrylic, methacrylic, maleic, itaconic, crotonic, vinylphthalic esters, or to the group of unsaturated urethanes such as, for example, acrylurethane, methacrylurethane, α-α′ dimethyl-isopropenyl-benzylurethane, allylurethane, or to the group of allylic or vinylic ethers, whether or not substituted, or again to the group of ethylenically unsaturated amides or imides,
R′ represents hydrogen or a hydrocarbonate radical having 1 to 40 atoms of carbon, and represents preferentially a hydrocarbonate radical having 1 to 12 carbon atoms, and very preferentially a hydrocarbonate radical having 1 to 4 carbon atoms,

or a mixture of several monomers of formula (J),
c) and possibly at least one monomer of the acrylamide or methacrylamide type and their mixtures, or again at least one non-hydrosoluble monomer such as the alkyl acrylates or methacrylates, the vinylics such as vinyl acetate, vinylpyrrolidone, styrene, alphamethylstyrene and their derivatives, or again at least one organofluorate or organosililate monomer, or their mixtures,
d) and possibly at least one monomer having at least two ethylenic unsaturations called in the remainder of the application a crosslinking monomer, or a mixture of several of these monomers.

In a particular manner, the use according to the invention of a dispersion and/or of suspension containing the said hydrosoluble polymers is also characterised in that the said polymers are obtained by controlled radical polymerisation of monomers chosen from:
a) at least one ionic monomer which is either:
i) anionic with ethylenic unsaturation and with a monocarboxylic function in the acid or acid-salt state chosen from among the ethylenic unsaturation monomers and with a monocarboxylic function such as acrylic or methacrylic acid, or again the diacid hemiesters such as the C₁ to C₄ monoesters of maleic or itaconic acids, or chosen from among the monomers with ethylenic unsaturation and with a dicarboxylic function in the acid or acid-salt state such as crotonic, isocrotonic, cinnamic, itaconic, maleic acid, or again the anhydrides of carboxylic acids, such as maleic anhydride, or chosen from among the monomers with ethylenic unsaturation and with a sulphonic function in the acid or acid-salt state such as 2-acrylamido-2-methyl-propane-sulphonic acid, sodium methallylsulphonate, vinyl sulphonic acid and styrene sulphonic acid, or again chosen from among the monomers with ethylenic unsaturation and with a phosphoric function in the acid or acid-salt state such as vinyl phosphoric acid, ethylene glycol methacrylate phosphate, propylene glycol methacrylate phosphate, ethylene glycol acrylate phosphate, propylene glycol acrylate phosphate and their ethoxylates, or again chosen from among the monomers with ethylenic unsaturation and with a phosphonic function in the acid or acid-salt state, such as vinyl phosphonic acid, or their mixtures, or
ii) cationic chosen from among N-[3-(dimethylamino) propyl]acrylamide or N-[3-(dimethylamino) propyl]methacrylamide, the unsaturated esters such as N-[2-(dimethylamino) ethyl]methacrylate, or N-[2-(dimethylamino) ethyl]acrylate, or from among the quaternary ammonium compounds such as [2-(methacryloyloxy)ethyl]trimethyl ammonium chloride or sulphate, [2-(acryloyloxy)ethyl]trimethyl ammonium chloride or sulphate, [3-(acrylamido) propyl]trimethyl ammonium chloride or sulphate, dimethyl diallyl ammonium chloride or sulphate, [3-(methacrylamido) propyl]trimethyl ammonium chloride or sulphate, or their mixtures, or
iii) a mixture of the abovementioned anionic and cationic monomers
b) and a least one monomer with a non-ionic ethylenic unsaturation of formula (I):

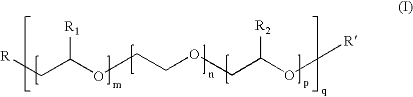

where:
m and p represent a number of alkylene oxide units of less than or equal to 150,
n represents a number of ethylene oxide units of less than or equal to 150,
q represents a whole number at least equal to 1 and such that 5≦(m+n+p)q ≦150, and preferentially such that 15≦(m+n+p)q≦120,
R₁ represents hydrogen or the methyl or ethyl radical,
R₂ represents hydrogen or the methyl or ethyl radical,
R represents a radical containing an unsaturated polymerisable function, belonging to the group of vinylics, or to the group of acrylic, methacrylic, maleic, itaconic, crotonic, vinylphthalic esters, or to the group of unsaturated urethanes such as, for example, acrylurethane, methacrylurethane, α-α′ dimethyl-isopropenyl-benzylurethane, allylurethane, or to the group of allylic or vinylic ethers, whether or not substituted, or again to the group of ethylenically unsaturated amides or imides,
R′ represents hydrogen or a hydrocarbonate radical having 1 to 40 atoms of carbon, and represents preferentially a hydrocarbonate radical having 1 to 12 carbon atoms, and very preferentially a hydrocarbonate radical having 1 to 4 carbon atoms,

or a mixture of several monomers of formula (I),
c) and possibly at least one monomer of the acrylamide or methacrylamide type and their mixtures, or again at least one non-hydrosoluble monomer such as the alkyl acrylates or methacrylates, the vinylics such as vinyl acetate, vinylpyrrolidone, styrene, alphamethylstyrene and their derivatives, or again at least one organofluorate or organosililate monomer chosen preferentially from among the molecules of formulae (IIa) or (IIb):

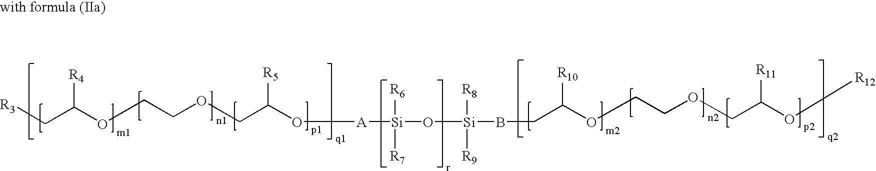

where:
m₁, p₁, m₂ and p₂ represent a number of alkylene oxide units of less than or equal to 150,
n₁ et n₂ represent a number of ethylene oxide units of less than or equal to 150,
q₁ and q₂ represent a whole number at least equal to 1 and such that 0≦(m₁+n₁+p₁)q₁≧150 and 0≧(m₂+n₂+p₂)q₂≦150,
r represents a number such that 1≦r≦200,
R₃ represents a radical containing an unsaturated polymerisable function, belonging to the group of vinylics, or to the group of acrylic, methacrylic, maleic, itaconic, crotonic, vinylphthalic esters, or to the group of unsaturated urethanes such as, for example, acrylurethane, methacrylurethane, α-α′ dimethyl-isopropenyl-benzylurethane, allylurethane, or to the group of allylic or vinylic ethers, whether or not substituted, or again to the group of ethylenically unsaturated amides or imides,
R₄, R₅, R₁₀ et R₁₁ represent hydrogen or the methyl or ethyl radical,
R₆, R₇, R₈ and R₉ represent linear or branched alkyl or aryl, or alkylaryl or arylalkyl groupings, having 1 to 20 carbon atoms, or their mixtures,
R₁₂ represents a hydrocarbonated radical having 1 to 40 carbon atoms,

A and B are groupings which may be present, which then represent a hydrocarbonated radical having 1 to 4 carbon atoms, with formula (IIb)

R-A-Si(OB)₃

where:
R represents a radical containing an unsaturated polymerisable function, belonging to the group of vinylics, or to the group of acrylic, methacrylic, maleic, itaconic, crotonic, vinylphthalic esters, or to the group of unsaturated urethanes such as, for example, acrylurethane, methacrylurethane, α-α′ dimethyl-isopropenyl-benzylurethane, allylurethane, or to the group of allylic or vinylic ethers, whether or not substituted, or again to the group of ethylenically unsaturated amides or imides,
A is a grouping which may be present, which then represents a hydrocarbonated radical having 1 to 4 carbon atoms,
B represents a hydrocarbonated radical having 1 to 4 carbon atoms,

or the mixture of several of these monomers,
d) and possibly at least one crosslinking monomer chosen from the group constituted by ethylene glycol dimethacrylate, trimethylolpropanetriacrylate, allyl acrylate, the allyl maleates, methylene-bis-acrylamide, methylene-bis-methacrylamide, tetrallyloxyethane, the triallylcyanurates, the allyl ethers obtained from polyols such as pentaerythritol, sorbitol, sucrose or others, or chosen from among the molecules of formula (III):

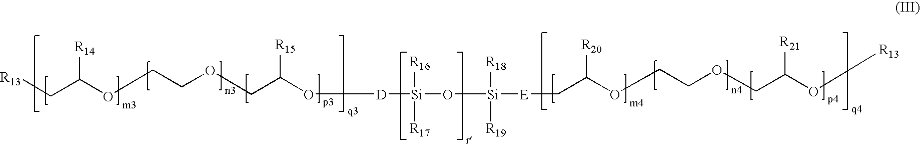

where:
m₃, p₃, m₄ and p₄ represent a number of alkylene oxide units of less than or equal to 150,
n₃ et n₄ represent a number of ethylene oxide units of less than or equal to 150,
q₃ and q₄ represent a whole number at least equal to 1 and such that 0≦(m₃+n₃+p₃)q₃≦150 and 0≦(m₄+n₄+p₄)q₄≦150,
r′ represents a number such that 1≦r′≦200,
R₁₃ represents a radical containing an unsaturated polymerisable function, belonging to the group of vinylics, or to the group of acrylic, methacrylic, maleic, itaconic, crotonic, vinylphthalic esters, or to the group of unsaturated urethanes such as, for example, acrylurethane, methacrylurethane, α-α′ dimethyl-isopropenyl-benzylurethane, allylurethane, or to the group of allylic or vinylic ethers, whether or not substituted, or again to the group of ethylenically unsaturated amides or imides,
R₁₄, R₁₅, R₂₀ and R₂₁ represent hydrogen or the methyl or ethyl radical,
R₁₆, R₁₇, R₁₈ and R₁₉ represent linear or branched alkyl or aryl, or alkylaryl or arylalkyl groupings, having 1 to 20 carbon atoms, or their mixtures,
D and E are groupings which may be present, which then represent a hydrocarbonated radical having 1 to 4 carbon atoms,

or the mixture of several of these monomers.

More specifically, the use according to the invention of a dispersion and/or of a suspension containing the said hydrosoluble polymers is characterised in that the said abovementioned polymers are constituted, expressed in weight, relative to the total weight of the monomers used:
a) from 2% to 98% and yet more particularly from 5% to 95% of a least one ionic monomer which is either:
i) an anionic monomer with ethylenic unsaturation and with a monocarboxylic function in the acid or acid-salt state chosen from among the ethylenic unsaturation monomers and with a monocarboxylic function such as acrylic or methacrylic acid, or again the diacid hemiesters such as the C₁ to C₄ monoesters of maleic or itaconic acids, or chosen from among the monomers with ethylenic unsaturation and with a dicarboxylic function in the acid or acid-salt state such as crotonic, isocrotonic, cinnamic, itaconic, maleic acid, or again the anhydrides of carboxylic acids, such as maleic anhydride, or chosen from among the monomers with ethylenic unsaturation and with a sulphonic function in the acid or acid-salt state such as 2-acrylamido-2-methyl-propane-sulphonic acid, sodium methallylsulphonate, vinyl sulphonic acid and styrene sulphonic acid, or again chosen from among the monomers with ethylenic unsaturation and with a phosphoric function in the acid or acid-salt state such as vinyl phosphoric acid, ethylene glycol methacrylate phosphate, propylene glycol methacrylate phosphate, ethylene glycol acrylate phosphate, propylene glycol acrylate phosphate and their ethoxylates, or again chosen from among the monomers with ethylenic unsaturation and with a phosphonic function in the acid or acid-salt state, such as vinyl phosphonic acid, or their mixtures, or
ii) a cationic monomer chosen from among N-[3-(dimethylamino) propyl]acrylamide or N-[3-(dimethylamino) propyl]methacrylamide, the unsaturated esters such as N-[2-(dimethyl amino) ethyl]methacrylate, or N-[2-(dimethylamino) ethyl]acrylate, or from among the quaternary ammonium compounds such as [2-(methacryloyloxy)ethyl]trimethyl ammonium chloride or sulphate, [2-(acryloyloxy)ethyl]trimethyl ammonium chloride or sulphate, [3-(acrylamido) propyl]trimethyl ammonium chloride or sulphate, dimethyl diallyl ammonium chloride or sulphate, [3-(methacrylamido) propyl]trimethyl ammonium chloride or sulphate, or their mixtures, or
iii) a mixture of the abovementioned anionic and cationic monomers,
b) from 2 to 98% and yet more particularly from 5% to 95% of a least one monomer with non-ionic ethylenic unsaturation of formula (I):

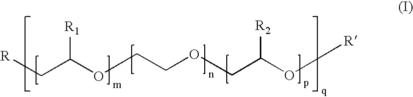

where:
m and p represent a number of alkylene oxide units of less than or equal to 150,
n represents a number of ethylene oxide units of less than or equal to 150,
q represents a whole number at least equal to 1 and such that 5≦(m+n+p)q≦150, and preferentially such that 15≦(m+n+p)q≦120,
R₁ represents hydrogen or the methyl or ethyl radical,
R₂ represents hydrogen or the methyl or ethyl radical,
R represents a radical containing an unsaturated polymerisable function, belonging to the group of vinylics, or to the group of acrylic, methacrylic, maleic, itaconic, crotonic, vinylphthalic esters, or to the group of unsaturated urethanes such as, for example, acrylurethane, methacrylurethane, α-α′ dimethyl-isopropenyl-benzylurethane, allylurethane, or to the group of allylic or vinylic ethers, whether or not substituted, or again to the group of ethylenically unsaturated amides or imides,
R′ represents hydrogen or a hydrocarbonate radical having 1 to 40 atoms of carbon, and represents preferentially a hydrocarbonate radical having 1 to 12 carbon atoms, and very preferentially a hydrocarbonate radical having 1 to 4 carbon atoms,

or a mixture of several monomers of formula (I),
c) from 0 to 50% of at least one monomer of the acrylamide or methacrylamide type and their mixtures, or again at least one non-hydrosoluble monomer such as the alkyl acrylates or methacrylates, the vinylics such as vinyl acetate, vinylpyrrolidone, styrene, alphamethylstyrene and their derivatives, or again at least one organofluorate or organosililate monomer chosen preferentially from among the molecules of formulae (IIa) or (IIb):

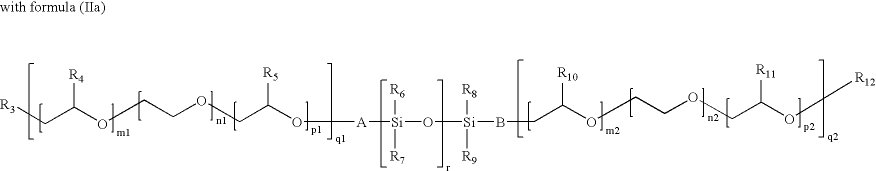

where:
m₁, p₁, m₂ and p₂ represent a number of alkylene oxide units of less than or equal to 150,
n₁ et n₂ represent a number of ethylene oxide units of less than or equal to 150,
q₁ and q₂ represent a whole number at least equal to 1 and such that 0≦(m₁+n₁+p₁)q₁≦150 and 0≦(m₂+n₂+p₂)q₂≦150,
r represents a number such that 1≦r≦200,
R₃ represents a radical containing an unsaturated polymerisable function, belonging to the group of vinylics, or to the group of acrylic, methacrylic, maleic, itaconic, crotonic, vinylphthalic esters, or to the group of unsaturated urethanes such as, for example, acrylurethane, methacrylurethane, α-α′ dimethyl-isopropenyl-benzylurethane, allylurethane, or to the group of allylic or vinylic ethers, whether or not substituted, or again to the group of ethylenically unsaturated amides or imides,
R₄, R₅, R₁₀ and R₁₁ represent hydrogen or the methyl or ethyl radical,
R₆, R₇, R₈ and R₉ represent linear or branched alkyl or aryl, or alkylaryl or arylalkyl groupings, having 1 to 20 carbon atoms, or their mixtures,
R₁₂ represents a hydrocarbonated radical having 1 to 40 carbon atoms,
A and B are groupings which may be present, which then represent a hydrocarbonated radical having 1 to 4 carbon atoms,

with formula (IIb)

R-A-Si(OB)₃

where:
R represents a radical containing an unsaturated polymerisable function, belonging to the group of vinylics, or to the group of acrylic, methacrylic, maleic, itaconic, crotonic, vinylphthalic esters, or to the group of unsaturated urethanes such as, for example, acrylurethane, methacrylurethane, α-α′ dimethyl-isopropenyl-benzylurethane, allylurethane, or to the group of allylic or vinylic ethers, whether or not substituted, or again to the group of ethylenically unsaturated amides or imides,
A is a grouping which may be present, which then represents a hydrocarbonated radical having 1 to 4 carbon atoms,
B represents a hydrocarbonated radical having 1 to 4 carbon atoms,

or the mixture of several of these monomers,
d) from 0 to 3% of at least one crosslinking monomer chosen from the group constituted by ethylene glycol dimethacrylate, trimethylolpropanetriacrylate, allyl acrylate, the allyl maleates, methylene-bis-acrylamide, methylene-bis-methacrylamide, tetrallyloxyethane, the triallylcyanurates, the allyl ethers obtained from polyols such as pentaerythritol, sorbitol, sucrose or others, or chosen from among the molecules of formula (III):

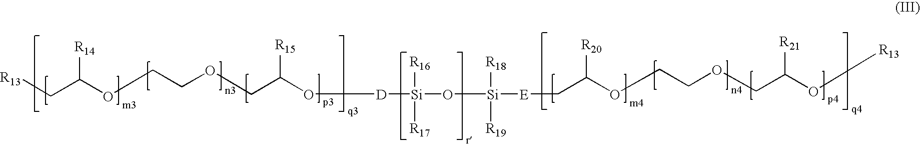

where:
m₃, p₃, m₄ and p₄ represent a number of alkylene oxide units of less than or equal to 150,
n₃ et n4 represent a number of ethylene oxide units of less than or equal to 150,
q₃ and q₄ represent a whole number at least equal to 1 and such that 0≦(m₃+n₃+p₃)q₃≦150 and 0≦(m₄+n₄+p₄)q₄≦150,
r′ represents a number such that 1≦r′≦200,
R₃ represents a radical containing an unsaturated polymerisable function, belonging to the group of vinylics, or to the group of acrylic, methacrylic, maleic, itaconic, crotonic, vinylphthalic esters, or to the group of unsaturated urethanes such as, for example, acrylurethane, methacrylurethane, α-α′ dimethyl-isopropenyl-benzylurethane, allylurethane, or to the group of allylic or vinylic ethers, whether or not substituted, or again to the group of ethylenically unsaturated amides or imides,
R₁₄, R₁₅, R₂₀ and R₂₁ represent hydrogen or the methyl or ethyl radical,
R₁₆, R₁₇, R₁₈ and R₁₉ represent linear or branched alkyl or aryl, or alkylaryl or arylalkyl groupings, having 1 to 20 carbon atoms, or their mixtures,
D and E are groupings which may be present, which then represent a hydrocarbonated radical having 1 to 4 carbon atoms,

or the mixture of several of these monomers

This use according to the invention of a dispersion and/or a suspension containing the said hydrosoluble polymers is also characterised in that the said polymers are obtained in the acid form and possibly distilled, and may be partially or totally neutralised by one or more agents such as the metal oxides or hydroxides, such as, for example, those of aluminium, and notably oxides or hydroxides of alkaline metals, such as, for example, sodium, potassium, lithium, or oxides or hydroxides of the alkaline earths such as, for example, calcium, magnesium, barium, or again the oxides or hydroxides of transition metals such as, for example, zinc, copper, iron, or again by ammonia or by the primary, secondary or tertiary aliphatic and/or cyclic amines such as, for example, stearylamine, the ethanolamines (mono-, di-, tri-ethanolamine), mono- and diethylamine, cyclohexamine, methylcyclohexylamine, amino methyl propanol, or morpholine.

According to another variant, the said polymers may also be before or after total or partial neutralisation reaction, treated and separated into several phases, according to static or dynamic processes known to the skilled man in the art, by one or more polar solvents belonging notably to the group constituted by water, methanol, ethanol, propanol, isopropanol, the butanols, acetone, tetrahydrofuran or their mixtures.

According to another variant, the said polymers may possibly be dried by one of the techniques well known to the skilled man in the art.

This use according to the invention of a dispersion and/or the suspension containing the said hydrosoluble polymers is also characterised in that the said polymers are hydrosoluble copolymers and have a structure of the statistic, block, comb, grafted or alternate type.

Depending on its use, the skilled man in the art will be able to adapt the molecular weight of the polymers used according to the invention.

This molecular weight is determined using the GPC (Gel Permeability Chromatography) method, using a liquid chromatography device of Waters™ brand fitted with two detectors, one of which combines dynamic diffusion of light with viscometry measured using a Viscotek™ viscometer, the other being a detector of refractometric concentration of brand Waters™.

This liquid chromatography equipment is fitted with steric exclusion columns suitably chosen by the skilled man in the art in order to separate the different molecular weights of the polymers studied.

The elution liquid phase is an aqueous phase.

This use is also characterised in that the mineral matter is chosen from among a pigment and/or a mineral filler, chosen from among natural or synthetic calcium carbonate, the dolomites, kaolin, talc, cement, gypsum, lime, magnesia, titanium dioxide, satin white, aluminium trioxide, or again aluminium trihydroxide, the silicas, mica and a mixture of these fillers one with another, such as talc-calcium carbonate mixtures, calcium carbonate-kaolin mixtures, or again mixtures of calcium carbonate with aluminium trihydroxide or aluminium trioxide, or again mixtures with synthetic or natural fibres, or again mineral costructures such as talc-calcium carbonate costructures or talc-titanium dioxide costructures, or their mixtures.

Preferentially, this use is characterised in that the said mineral matter is natural or synthetic calcium carbonate, and more specifically a natural calcium carbonate chosen from among marble, calcite, chalk or their mixtures.

Another object of the invention concerns dispersions and/or water-based suspensions of mineral matter, characterised in that they contain:
water,
at least one mineral matter,
at least one hydrosoluble polymer obtained by a process of controlled radical polymerisation using an alcoxyamine of formula (A)
and possibly at least one dispersing agent and/or at least one grinding aid agent different from the abovementioned polymer,
   and in that they are obtained by the process according to the invention.

These dispersions and/or suspensions are also characterised in that they contain from 0.05% to 5%, and preferentially from 0.1% to 3%, by dry weight of at least one hydrosoluble polymer obtained by a process of controlled radical polymerisation using an alcoxyamine of formula (A), relative to the dry weight of mineral matter.

Another object of the invention concerns water-based formulations, and notably paper coatings, mass fillers, water-based paints and cosmetic formulations, characterised in that they contain:
water,
at least one mineral matter,
at least one hydrosoluble polymer obtained by a process of controlled radical polymerisation using an alcoxyamine of formula (A),
and possibly at least one dispersing agent and/or at least one grinding aid agent different from the abovementioned polymer,
   and in that they are obtained by the process according to the invention.

These formulations are also characterised in that they contain from 0.05% to 5%, and preferentially from 0.1% to 3% by dry weight of at least one hydrosoluble polymer obtained by a process of controlled radical polymerisation using an alcoxyamine of formula (A), relative to the total weight of the formulation.

Another object of the invention concerns dry powders of mineral matter, characterised in that they contain:
at least one mineral matter,
and at least one hydrosoluble polymer obtained by a process of controlled radical polymerisation using an alcoxyamine of formula (A).

These dry powders are also characterised in that they contain from 0.05% to 5%, and preferentially from 0.1% to 3% by weight of at least one hydrosoluble polymer obtained by a process of controlled radical polymerisation using an alcoxyamine of formula (A), relative to the total weight of the powder.

A final object of the invention concerns the films resulting from the drying of a paper coating, films resulting from the drying of a water-based paint formulation, films resulting from the drying of a water-based cosmetic formulation, and plastics and filled papers, characterised in that they contain:
at least one mineral matter,
and at least one hydrosoluble polymer obtained by a process of controlled radical polymerisation using an alcoxyamine of formula (A).

The scope and interest of the invention will be better appreciated through the following examples, which are by no means limitative.

### EXAMPLE 1

This example illustrates the process whereby hydrosoluble polymers are obtained, use of which in the subsequent examples forms the object of the invention.

In tests n^{o}1 to 12, a process of controlled radical polymerisation is implemented using methods well known to the skilled man in the art, using as an initiator of polymerisation alcoxyamine of formula (A′):

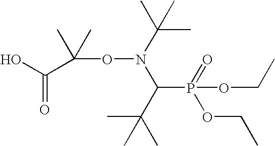

Controlled structure hydrosoluble polymers are then obtained, the monomer composition and molecular weight of which (measured according to the method previously described and expressed in g/mole) are given in tests n^{o}1 to 12.

### Test N^{o}1

This test leads to the obtaining, using a process of controlled radical polymerisation using as an initiator for polymerisation alcoxyamine of formula (A′), a hydrosoluble polymer of composition by weight of, relative to the total mass of monomers used:
18.4% methacrylic acid,
79.6% methoxy polyethylene glycol methacrylate of molecular mass 2000,
1.5% butoxypoly(oxyethylene oxypropylene) methacrylate containing 10 units of oxyethylene and 11 units of oxypropylene,
0.5% ethylene glycol dimethacrylate, of which the previously described GPC analysis indicates to us an average molecular mass by weight Mw of 40,000 g/mole.

### Test N^{o}2

This test leads to the obtaining, using a process of controlled radical polymerisation using as an initiator of polymerisation alcoxyamine of formula (A′), a hydrosoluble polymer of composition by weight of, relative to the total mass of monomers used:
18.5% methacrylic acid,
81.5% methoxy polyethylene glycol methacrylate of molecular mass 2000,
   of which the previously described GPC analysis indicates to us an average molecular mass by weight Mw of 66,100 g/mole.

### Test N^{o}3

This test leads to the obtaining, using a process of controlled radical polymerisation using as an initiator of polymerisation alcoxyamine of formula (A′), a hydrosoluble polymer of composition by weight of, relative to the total mass of monomers used:
18.5% methacrylic acid,
81.5% methoxy polyethylene glycol methacrylate of molecular mass 2000,
   of which the previously described GPC analysis indicates to us an average molecular mass by weight Mw of 33,300 g/mole.

### Test N^{o}4

This test leads to the obtaining, using a process of controlled radical polymerisation using as an initiator of polymerisation alcoxyamine of formula (A′), a hydrosoluble polymer of composition by weight of, relative to the total mass of monomers used:
18.4% methacrylic acid,
81.1% methoxy polyethylene glycol methacrylate of molecular mass 2000,
0.5% of a sililated monomer of formula R-A-Si(OB)₃, where R designates the methacrylate group, A designates the propyl radical and B designates the methyl radical,
   of which the previously described GPC analysis indicates to us an average molecular mass by weight Mw of 32,470 g/mole.

### Test N^{o}5

This test leads to the obtaining, using a process of controlled radical polymerisation using as an initiator of polymerisation alcoxyamine of formula (A′), a hydrosoluble polymer of composition by weight of, relative to the total mass of monomers used:
7.5% methacrylic acid,
91.0% methoxy polyethylene glycol methacrylate of molecular mass 5000,
1.5% butoxypolyoxypropylene hemimaleate containing 19 units of oxypropylene.
   of which the previously described GPC analysis indicates to us an average molecular mass by weight Mw of 51,900 g/mole.

### Test N^{o}6

This test leads to the obtaining, using a process of controlled radical polymerisation using as an initiator of polymerisation alcoxyamine of formula (A′), a hydrosoluble polymer of composition by weight of, relative to the total mass of monomers used:
18.5% methacrylic acid,
81.5% methoxy polyethylene glycol methacrylate of molecular mass 2000,
   of which the previously described GPC analysis indicates to us an average molecular mass by weight Mw of 105,730 g/mole.

### Test N^{o}7

This test leads to the obtaining, using a process of controlled radical polymerisation using as an initiator of polymerisation alcoxyamine of formula (A′), a hydrosoluble polymer of composition by weight of, relative to the total mass of monomers used:
49.0% methacrylic acid,
51.0% methoxy polyethylene glycol methacrylate of molecular mass 2000,
   of which the previously described GPC analysis indicates to us an average molecular mass by weight Mw of 25,430 g/mole.

### Test N^{o}8

This test leads to the obtaining, using a process of controlled radical polymerisation using as an initiator of polymerisation alcoxyamine of formula (A′), a hydrosoluble polymer of composition by weight of, relative to the total mass of monomers used:
7.5% methacrylic acid,
92.5% methoxy polyethylene glycol methacrylate of molecular mass 2000,
   of which the previously described GPC analysis indicates to us an average molecular mass by weight Mw of 25,570 g/mole.

### Test N^{o}9

This test leads to the obtaining, using a process of controlled radical polymerisation using as an initiator of polymerisation alcoxyamine of formula (A′), a hydrosoluble polymer of composition by weight of, relative to the total mass of monomers used:
18.5% methacrylic acid,
81.5% methoxy polyethylene glycol methacrylate of molecular mass 2000,
   of which the previously described GPC analysis indicates to us an average molecular mass by weight Mw of 28,330 g/mole.

### Test N^{o}10

This test leads to the obtaining, using a process of controlled radical polymerisation using as an initiator of polymerisation alcoxyamine of formula (A′), a hydrosoluble polymer of composition by weight of, relative to the total mass of monomers used:
18.5% methacrylic acid,
81.5% methoxy polyethylene glycol methacrylate of molecular mass 2000,
   of which the previously described GPC analysis indicates to us an average molecular mass by weight Mw of 25,330 g/mole.

### Test N^{o}11

This test leads to the obtaining, using a process of controlled radical polymerisation using as an initiator of polymerisation alcoxyamine of formula (A′), a hydrosoluble polymer of composition by weight of, relative to the total mass of monomers used:
18.5% methacrylic acid,
81.5% methoxy polyethylene glycol methacrylate of molecular mass 2000,
   of which the previously described GPC analysis indicates to us an average molecular mass by weight Mw of 25,470 g/mole.

### Test N^{o}12

This test leads to the obtaining, using a process of controlled radical polymerisation using as an initiator of polymerisation alcoxyamine of formula (A′), a hydrosoluble polymer of composition by weight of, relative to the total mass of monomers used:
18.5% methacrylic acid,
81.5% methoxy polyethylene glycol methacrylate of molecular mass 2000,
   of which the previously described GPC analysis indicates to us an average molecular mass by weight Mw of 23,000 g/1 mole.

### EXAMPLE 2

This example illustrates the use according to the invention of controlled structure hydrosoluble polymers obtained by a process of controlled radical polymerisation using, as an initiator of polymerisation, a particular alcoxyamine of formula (A′), together with the use according to the invention of water-based dispersions of mineral matter containing the said polymers, in a process of manufacture of dry products containing mineral matter, as agents improving the opacity and brightness of the said dry products.

In this example, the process of manufacture of the said dry products is characterised in that it includes a stage of grinding in water without the grinding aid agent, and a stage of concentration with the use of the said polymers, leading to the obtaining of a water-based dispersion of mineral matter containing the said polymers. The said dispersion allows paper coatings to be formulated which lead, after drying, to the obtaining of dry coverings, the opacity and brightness of which are improved.

In this example, the dry coverings concerned are dry films which result from the drying of the said paper coatings which have been applied on to sheets of paper.

### Preparation of the Water-Based Dispersions of Mineral Matter, of the Paper Coatings Containing the Said Dispersions and of the Papers Coated Using the Said Dispersions.

In tests n^{o}13 to 16, one begins by grinding a calcium carbonate which is a marble originating from Norway, using a wet method, and without a grinding aid agent, according to the methods well known to the skilled man in the art.

The solid content of the mineral matter suspension thus obtained is then equal to 20% (by dry weight of mineral matter relative to the total dry weight of the suspension).

The percentages by weight of the particles the diameter of which is less than 1 μm and than 2 μm is then determined, using a Sedigraph™ 5100 device sold by the company MICROMERITICS™, noted respectively %<2 μm and %<1 μm.

The suspension obtained is then concentrated in the presence of a dispersing agent of the prior art, or by use of a polymer according to the invention.

A dispersion of mineral matter is then obtained, the solid content of which is then measured, noted SC, and expressed as a percentage by dry weight of mineral matter relative to the total weight of the suspension, the granulometric characteristics of the latter as determined above, %<2 in and %<1 μm, remaining unchanged.

The dispersion thus obtained is then mixed with a styrene-acrylic binder sold under the name Acronal™ S360D by the company BASF™, in a proportion of 100 grams by dry weight of calcium carbonate for 12 parts of binder in its initial state.

The paper coatings are then used to coat sheets of paper.

Before being coated, each sheet of paper is subjected to a light radiation of wavelength equal to 457 nm on a black plate using a Elrepho™ 3000 spectrophotometer from Datacolor™ (Switzerland) to determine the reflectance factor R_{b} of the non-coated paper on a black background.

With the preparation thus obtained, a polypropylene-based plastic paper sold under the name Synteape™ by the company AJO-WIGGNS™ is thus coated using a laboratory coating machine of the Hand Coater™ type, model KC202, to obtain different coating weights between 5 and 50 m²/g.

The samples are dried at 90° C. for 10 minutes in an oven.

Measurement of Optical Properties in Relation to the Dry Films Resulting from the Drying of the Paper Coatings Applied on to the Papers.

For each of the tests n^{o}13 to 16, different measurements of optical properties for the dry films resulting from the drying of the paper coatings applied to the papers according to the invention and according to the prior art are then undertaken.

Determination of the Light Diffusion Factor S (m²/kg) Before Calendering.

Each sheet of paper coated with different coating weights between 5 and 50 g/m² is subject to a light radiation of wavelength equal to 457 nm n using a Elrepho™ 3000 spectrophotometer sold by the company DATACOLOR™ on a black plate to determine the reflectance factor of the coated paper on a black background R₀ and on a pile of at least 10 sheets of non-coated paper to determine the reflectance factor of the coated papers on a white background R₁, where r is the reflectance factor of the pile of sheets of non-coated paper.

The reflectance factor R_{sc} of the coat alone, on a black background, is then determined by the formula:

$${R}_{sc} = \frac{{R}_{1} \cdot {R}_{b} \text{-} {R}_{0} \cdot r}{( {R}_{1} \text{-} {R}_{0} ) \cdot {rR}_{b} + {R}_{b} - r} \qquad ( 1 )$$

together with the transmission T_{sc} of the coat

$${T}_{sc}^{2} = \frac{( {R}_{0} \text{-} {R}_{sc} ) ( 1 - {R}_{sc} {R}_{b} )}{{R}_{b}} \qquad ( 2 )$$

From these two quantities, it is possible to calculate a theoretical reflectance value L, for a layer of infinite thickness given by the formula:

$$\frac{1 - {T}_{sc}^{2} + {R}_{sc}^{2}}{{R}_{C}} = \frac{1 + {R}_{∞}^{2}}{{R}_{∞}} \qquad ( 3 )$$

Using this formula, it is possible to calculate the light diffusion factor S for each coating weight, given that, for a coating weight P, we have:

$$\begin{matrix}S . P . = \frac{1}{b} {coth}^{- 1} \frac{( 1 \text{-} {aR}_{sc} )}{{bR}_{sc}} \\ With : a = 0 , 5 ( \frac{1}{{R}_{∞}} + {R}_{∞} ) \\ b = 0 , 5 ( \frac{1}{{R}_{∞}} + {R}_{∞} )\end{matrix}$$

This light diffusion factor S is graphed as a function of the coating weight and the value S for a coating weight equal to 20 g/m² is determined by interpolation.

Measurement of Brightness after Calendering

The different samples are then calendered at 90° C. under 60 bar pressure, using a calendering device of type RK 22 HU sold by the company DR DRAMISCH™ & CO.

The samples are dried for 10 minutes in an oven at 90° C.

Using these samples, a measurement of the unique brightness called the “75° TAPPI brightness measurement” is then determined, and noted B₇₅, according to the procedure described in norm TAPPI T480 os-78, using a laboratory glossmeter of type LGDL-05/2 sold by the company LEHMANN MESSTECHNIK™.

### Test N^{o}13

This test illustrates the prior art and uses 1.50% by dry weight of a copolymer of the prior art containing 70% by weight of acrylic acid and 30% by weight of maleic anhydride, relative to the dry weight of mineral matter, during the process of concentration of the said mineral matter.

### Test N^{o}14

This test illustrates the invention and uses 1.00% by dry weight of the polymer according to test n^{o}11 relative to the dry weight of mineral matter, during the process of concentration of the said mineral matter.

### Test N^{o}15

This test illustrates the invention and uses 1.20% by dry weight of the polymer according to test n^{o}11 relative to the dry weight of mineral matter, during the process of concentration of the said mineral matter.

### Test N^{o}16

This test illustrates the invention and uses 1.50% by dry weight of the polymer according to test n^{o}1 relative to the dry weight of mineral matter, during the process of concentration of the said mineral matter.

For tests n^{o}13 to 16, table 1 indicates:
the value of the solid content noted SC (as a % by dry weight of mineral matter relative to the total dry weight of the dispersion in question) for each dispersion;
the percentage by weight of particles the diameter of which is less than 1 μm and than 2 μm for each dispersion, respectively noted %<1 μn and %<2 μm;
measurements of the brightness after calendering for each coated paper, noted B₇₅;
measurements of the light diffusion factor for each coated paper before calendering, noted S (m²/kg).

TABLE 1
characteristics of the water-based dispersions of mineral
matter and optical properties of the dry coverings.

| | | Characteristics of the | | | | |
|---|---|---|---|---|---|---|
| | | dispersions of mineral | | | Optical properties of | |
| Test | Prior art/ | matter | | | the dry coverings | |
| n° | Invention | SC (%) | % <2 μm | % <1 μm | S (m²/kg) | B₇₅ |
| 13 | Prior art | 70.1 | 96.6 | 71.5 | 146 | 66 |
| 14 | Invention | 69.8 | 96.2 | 68.4 | 184 | 68 |
| 15 | Invention | 69.6 | 96.1 | 69.8 | 200 | 68 |
| 16 | Invention | 69.5 | 96.5 | 70.2 | 192 | 68 |

A reading of table 1 demonstrates that the characteristics of the water-based dispersions according to the invention are similar to those of the dispersion of the prior art, in terms of solid content and granulometry.

Conversely, the opacity, in terms of measurement of the light diffusion factor S, and the brightness, in terms of measurement of the factor B₇₅, of the dry films resulting from the drying of the paper coatings applied on to the papers are better in the case of the coatings which use the dispersions obtained by the process according to the invention.

This effect is particularly remarkable since the dispersions according to the invention use a lesser quantity of polymer compared to the prior art.

This example thus demonstrates that the use according to the invention of the said polymers, together with the use according to the invention of the said dispersions containing the said polymers, enable the opacity and the brightness of the dry coverings which are dry films resulting from the drying of paper coatings containing the dispersions and the polymers according to the invention to be improved substantially.

### EXAMPLE 3

This example illustrates the use according to the invention of controlled structure hydrosoluble polymers obtained by a process of controlled radical polymerisation using, as an initiator of polymerisation, a particular alcoxyamine of formula (A′), together with the use according to the invention of water-based dispersions of mineral matter containing the said polymers, in a process of manufacture of dry products containing mineral matter, as agents improving the opacity and brightness of the said dry products.

In this example, the process of manufacture of the said dry products is characterised in that it includes a stage of grinding in water and a stage of concentration in water of the said mineral matter: both stages take place with use of the polymers according to the invention or according to the prior art. It leads to the obtaining of a water-based dispersion of mineral matter containing the said polymers, which enable paper coatings to be manufactured.

The manufacturing process is also characterised in that it enables dry coverings to be obtained, resulting from the drying of the said paper coatings previously applied on to papers.

Preparation of the Water-Based Dispersions of Mineral Matter, of the Paper Coatings Containing them, and of the Papers Coated Using the Said Dispersions.

In tests n^{o}17 to 20, one begins by grinding using a wet method, and using the methods well known to the skilled man in the art, a calcium carbonate sold by the company OMYA™ under the name Omyacarb™ 10 AV, by use of a polymer of the prior art or of a polymer according to the invention.

The solid content of the mineral matter suspension thus obtained, noted SC, (by % of dry weight of mineral matter relative to the total dry weight of the suspension), is then determined.

The percentage by weight of the particles the diameter of which is less than 1 μm and than 2 μm is then determined, using a Sedigraph™ 5100 device sold by the company MICROMERITICS™, noted %<1 μm and %<2 μm.

The suspension obtained is then concentrated in the presence of a dispersing agent of the prior art, or by use of a polymer according to the invention: this is the same polymer as the one used during the grinding stage.

A dispersion of mineral matter is then obtained, the solid content of which is then measured, noted SC₂ (as a % by dry weight of mineral matter relative to the total weight of the suspension), the granulometric characteristics of the latter as determined above remaining unchanged.

The dispersion thus obtained is then mixed with a styrene-acrylic binder sold under the name Acronal™ S360D by the company BASF™, in a proportion of 100 grams by dry weight of calcium carbonate for 12 parts of binder in its initial state.

With the preparation thus obtained, a polypropylene-based plastic paper sold under the name Synteape™ by the company ARJO-WIGGINS™ is thus coated using a laboratory coating machine of the Hand Coater™ type, model KC202, to obtain different coating weights between 5 and 50 g/m².

The samples are dried at 90° C. for 10 minutes in an oven.

Measurement of Optical Properties in Relation to the Dry Films Resulting from the Drying of the Paper Coatings Applied on to the Papers.

In the same manner as that described for example 2, the values of the light diffusion factor S (m²/kg) are then determined before calendering, together with the 75° TAPPI brightness, noted B₇₅, measured after calendering of the coated sheet of paper.

### Test N^{o}17

This test illustrates the prior art and uses 0.25% by dry weight, relative to the dry weight of mineral matter, of a copolymer consisting of 70% acrylic acid and 30% maleic anhydride (by % of weight of monomers), during the grinding stage, and 0.45% by dry weight of the same copolymer relative to the dry weight of mineral matter during the concentration stage.

### Test N^{o}18

This test illustrates the invention and uses 0.25% by dry weight of polymer according to test n^{o}3 relative to the dry weight of mineral matter, during the grinding stage, and 0.45% by dry weight of the same copolymer relative to the dry weight of mineral matter during the concentration stage.

### Test N^{o}19

This test illustrates the invention and uses 0.25% by dry weight of polymer according to test n^{o}2 relative to the dry weight of mineral matter, during the grinding stage, and 0.45% by dry weight of the same copolymer relative to the dry weight of mineral matter during the concentration stage.

### Test N^{o}20

This test illustrates the invention and uses 0.15% by dry weight of polymer according to test n^{o}3 relative to the dry weight of mineral matter, during the grinding stage, and 0.45% by dry weight of the same copolymer relative to the dry weight of mineral matter during the concentration stage.

For tests n^{o}17 to 20, table 2 indicates:
the value of the solid content noted SC₁ (as a % by dry weight of mineral matter relative to the total dry weight of the dispersion in question) for each suspension obtained after the grinding stage;
the value of the solid content noted SC₂ (as a % by dry weight of mineral matter relative to the total dry weight of the dispersion in question) for each dispersion obtained after the concentration stage;
the percentage by weight of particles the diameter of which is less than 1 μm and than 2 μm for each dispersion, respectively noted %<1 μm and %<2 μm;
measurements of the brightness after calendering for each coated paper, noted B₇₅;
measurements of the light diffusion factor S (m²/kg) for each coated paper before calendering.

TABLE 2
characteristics of the dispersions and of the water-based suspensions
of mineral matter and optical properties of the dry coverings.

| | | Characteristics of the dispersions and of | | | | Optical properties of | |
|---|---|---|---|---|---|---|---|
| Test | Prior art/ | the suspensions of mineral matter | | | | the dry coverings | |
| n° | Invention | SC₁ (%) | SC₂ (%) | % <2 μm | % <1 μm | S (m²/kg) | B₇₅ |
| 17 | Prior art | 35.0 | 71.0 | 91.0 | 60.0 | 144 | 66 |
| 18 | Invention | 35.0 | 70.8 | 92.2 | 60.2 | 153 | 70 |
| 19 | Invention | 35.0 | 69.6 | 90.7 | 58.2 | 147 | 68 |
| 20 | Invention | 30.0 | 70.5 | 91.4 | 61.7 | 145 | 68 |

A reading of table 2 demonstrates that the characteristics of the water-based dispersions according to the invention are similar to those of the dispersion of the prior art, in terms of solid content SC₂ and granulometry.

Conversely, the opacity, in terms of measurement of the light diffusion factor S, and the brightness, in terms of measurement of factor B₇₅, of the dry films resulting from the drying of the paper coatings containing the said dispersions, is higher in the process according to the invention.

This example thus demonstrates that the use according to the invention of the said polymers, together with the use according to the invention of the said dispersions containing the said polymers, enable the opacity and the brightness of the dry films obtained by drying of paper coatings containing the dispersions and the polymers according to the invention to be improved substantially.

### EXAMPLE 4

This example illustrates the use according to the invention of controlled structure hydrosoluble polymers obtained by a process of controlled radical polymerisation using, as an initiator of polymerisation, a particular alcoxyamine of formula (A′), together with the use of the water-based suspensions containing the said polymers, in a process of manufacture of dry products containing mineral matter, as agents improving the brightness and whiteness of the said dry products.

In this example, the process of manufacture of the said dry products is characterised in that it includes the stage of grinding in water of the mineral matter, with use of a polymer of the prior art or with use of polymers according to the invention, leading to the obtaining of a water-based suspension of mineral matter. This suspension enables a paper coating to be manufactured.

The manufacturing process is also characterised in that it applies to the manufacture of dry coverings which are dry films resulting from the drying of the said paper coatings which have been previously applied on to papers, which are papers coated using paper coatings containing the said suspensions.

### Preparation of Water-Based Suspensions of Mineral Matter.

In tests n^{o}21 to 24, one begins by grinding using a wet method, and using the methods well known to the skilled man in the art, a calcium carbonate which is a calcite from Orgon (France), by use of a polymer of the prior art or of a polymer according to the invention.

The solid content, noted SC, as a percentage by dry weight of mineral matter relative to the total dry weight of the suspension, and the percentage by weight of particles the diameter of which is less than 1 μm, noted %<1 μm, using a Sedigraph™ 5100 device sold by the company MICROMERITICS™, are then determined.

### Preparation of the Paper Coatings

A paper coating is then produced, containing:
68% by dry weight of mineral matter, relative to the total weight of the paper coating, introduced through the adequate quantity of suspension of mineral matter previously prepared;
0.4% by dry weight relative to the dry weight of mineral matter, of Finnfix™ 10, which is a cellulose carboxymethyl sold by the company METSA SERLA™;
0.4% by dry weight relative to the dry weight of mineral matter, of Mowiol™ 6.98, which is a polyvinylic alcohol sold by the company CLARIANT™;
11.0% by dry weight relative to the dry weight of mineral matter, of DL966, which is a latex sold by the company DOW™;
0.6% by dry weight relative to the dry weight of mineral matter, of Blancophor™ P, which is an optical brightener sold by the company CIBA™;
   The pH of the paper coating is adjusted to 8.5 through the addition of sodium hydroxide.

### Preparation of Coated Papers.

With the paper coatings obtained, a pre-coated support paper, the grammage of which is equal to 76 g/m², is then coated, using a laboratory coating machine of the Hand Coater™ type, model KC202, in order to obtain a coat weight equal to 10 g/m².

The samples are dried for 10 minutes in an oven at 90° C.

Measurement of Optical Properties in Relation to the Dry Films Resulting from the Drying of the Paper Coatings Applied on to the Papers.

The coated paper is then calendered at 80° C. under 40 bar pressure, using a calendering device of type RK 22 HU sold by the company DR DRAMISCH™ & CO.

On the coated and calendered sheets of paper, a measurement of the 75° TAPPI brightness is then made, noted B₇₅, according to norm TAPPI T480 os-78, using a laboratory glossmeter of type LGDL-05/2 sold by the company LEHMANN MESSTECHNIK™.

On the coated and calendered sheets of paper, a whiteness measurement is made, noted WCIE according to norm ISO/FDIS 11475.

### Test N^{o}21

This test illustrates the prior art and uses 1.35% by weight, relative to the dry weight of mineral matter, of a polymer of the prior art, which is a homopolymer of acrylic acid neutralised by a mixture of 50% sodium hydroxide and 50% magnesium hydroxide (in molar equivalence), during the grinding stage.

### Test N^{o}22

This test illustrates the prior art and uses during the grinding stage 1.35% by weight, relative to the dry weight of mineral matter, of a polymer of the prior art which is obtained by traditional radical polymerisation, and which consists, in terms of percentage by weight, of 81.5% methoxy polyethylene glycol methacrylate of molecular weight 2000, 4.9% methacrylic acid, and 13.6% acrylic acid.

### Test N^{o}23

This test illustrates the invention and uses 1.35% by weight, relative to the dry weight of mineral matter, of a polymer according to test n^{o}1, during the grinding stage.

### Test N^{o}24

This test illustrates the invention and uses 1.35% by weight, relative to the dry weight of mineral matter, of a polymer according to test n^{o}5, during the grinding stage.

For tests n^{o}21 to 24, table 3 indicates:
the value of the solid content noted SC (as a % by dry weight of mineral matter relative to the total dry weight of the suspension in question) for each suspension obtained after the grinding stage;
the percentage by weight of particles the diameter of which is less than 1 μm for each suspension, noted %<1 μm;
measurements of the brightness after calendering for each coated paper, noted B₇₅;
measurements of brightness after calendering for each coated paper, noted WCIE.

TABLE 3
characteristics of the water-based suspensions of mineral
matter and optical properties of the dry coverings.

| | | Characteristics of the | | | |
|---|---|---|---|---|---|
| | | suspensions of mineral | | Optical properties of | |
| Test | Prior art/ | matter | | the dry coverings | |
| n° | Invention | SC (%) | % <1 μm | B₇₅ | WCIE |
| 21 | Prior art | 74.6 | 80.7 | 75 | 113 |
| 22 | Prior art | 74.1 | 79.9 | 78 | 115 |
| 23 | Invention | 74.3 | 81.8 | 79 | 118 |
| 24 | Invention | 74.0 | 81.0 | 81 | 120 |

A reading of table 3 demonstrates that the characteristics of the water-based suspensions according to the invention are similar to those of the suspensions according to the prior art, in terms of solid content SC and granulometry.

Conversely, the brightness, in terms of measurement of factor B₇₅, and the whiteness, in terms of measurement of factor WCIE, of the dry coverings resulting from the drying of the paper coatings, is higher for the papers coated according to the invention.

This example thus demonstrates that the use according to the invention of the said polymers, together with the use according to the invention of the said suspensions containing the said polymers, enable the brightness and whiteness of the dry coverings which are dry films resulting from the drying of paper coatings containing the suspensions and the polymers according to the invention to be improved substantially.

### EXAMPLE 5

This example illustrates the use according to the invention of controlled structure hydrosoluble polymers obtained by a process of controlled radical polymerisation using, as an initiator of polymerisation, a particular alcoxyamine of general formula (A′), together with the use of the water-based suspensions containing the said polymers, in a process of manufacture of dry products containing mineral matter, as agents improving the opacity of the said dry products.

In this example, the process of manufacture of the said dry products is characterised in that it includes the stage of grinding in water of the mineral matter, with use of a polymer of the prior art or with use of polymers according to the invention, leading to the obtaining of a water-based suspension of mineral matter. This suspension enables a paper coating to be manufactured.

The manufacturing process is also characterised in that it enables dry coverings, which are dry films resulting from the drying of the said paper coatings which have been previously applied on to papers, to be obtained.

### Preparation of Water-Based Suspensions of Mineral Matter.

In tests n^{o}25 to 28, one begins by grinding using a wet method, and using the methods well known to the skilled man in the art, a calcium carbonate which is a calcite from Orgon (France), by use of a polymer of the prior art or of a polymer according to the invention.

The solid content, noted SC, as a percentage by dry weight of mineral matter relative to the total dry weight of the suspension, and the percentage by weight of particles the diameter of which is less than 1 μm, noted %<1 μm, using a Sedigraph™ 5100 device sold by the company MICROMERITICS™, are then determined.

### Preparation of the Paper Coatings

A paper coating is then produced using the same method as that described during example 2.

### Preparation of Coated Papers.

Coated papers are produced using the same method as that described for example 2.

The samples are dried for 10 minutes in an oven at 90° C.

Measurement of the Opacity of the Dry Films Resulting from the Drying of the Paper Coatings Applied on to the Papers.

The coated papers are then calendered using the same method as that described during example 2.

The value of the light diffusion factor S (m²/kg) is then determined before calendering, using the same method as that described in example 2.

### Test N^{o}25

This test illustrates the prior art and uses 1.35% by weight, relative to the dry weight of mineral matter, of a polymer of the prior art, which is a homopolymer of acrylic acid neutralised by a mixture of 50% sodium hydroxide and 50% magnesium hydroxide (in molar equivalence), during the grinding stage.

This test differs from test n^{o}21 in that the grinding operation has been stopped so as to obtain a solid content equal to 73.2% by dry weight of mineral matter relative to the total dry weight of the suspension.

### Test N^{o}26

This test illustrates the prior art and uses during the grinding stage 1.35% by weight, relative to the dry weight of mineral matter, of a polymer of the prior art which is obtained by traditional radical polymerisation, and which consists, in terms of percentage by weight, of 81.5% methoxy polyethylene glycol methacrylate of molecular weight 2000, 4.9% methacrylic acid, and 13.6% acrylic acid.

This test differs from test n^{o}22 in that the grinding operation has been stopped so as to obtain a solid content equal to 72.9% by dry weight of mineral matter relative to the total dry weight of the suspension.

### Test N^{o}27

This test illustrates the invention and uses 1.35% by weight, relative to the dry weight of mineral matter, of a polymer according to test n^{o}7, during the grinding stage.

### Test N^{o}28

This test illustrates the invention and uses 1.35% by weight, relative to the dry weight of mineral matter, of a polymer according to test n^{o}8, during the grinding stage.

For tests n^{o}25 to 28, table 4 indicates:
the value of the solid content noted SC (as a % by dry weight of mineral matter relative to the total dry weight of the suspension in question) for each suspension obtained after the grinding stage;
the percentage by weight of particles the diameter of which is less than 1 μm for each suspension, noted %<1 μm;
the value of the light diffusion factor S (m²/kg);

TABLE 4
characteristics of the water-based suspensions of
mineral matter and opacity of the dry coverings.

| | | Characteristics of the | | |
|---|---|---|---|---|
| | | suspensions of mineral | | Optical property of |
| Test | Prior art/ | matter | | the dry coverings |
| n° | Invention | SC (%) | % <1 μm | S (m²/kg) |
| 25 | Prior art | 73.2 | 80.3 | 72 |
| 26 | Prior art | 72.9 | 79.3 | 82 |
| 27 | Invention | 73.0 | 80.1 | 112 |
| 28 | Invention | 73.7 | 80.6 | 127 |

A reading of table 4 demonstrates that the characteristics of the water-based suspensions according to the invention are similar to those of the suspensions according to the prior art, in terms of solid content SC and granulometry.

Conversely, the opacity in terms of measurement of the light diffusion factor S of the dry films resulting from the drying of the paper coatings containing the suspension according to the invention is much better than in the case of the prior art

This example thus demonstrates that the use according to the invention of the said polymers, together with the use according to the invention of the said suspensions containing the said polymers, enable the opacity of the dry films resulting from the drying of paper coatings containing the dispersions and the polymers according to the invention to be improved substantially.

### EXAMPLE 6

This example illustrates the use according to the invention of controlled structure hydrosoluble polymers obtained by a process of controlled radical polymerisation using, as an initiator of polymerisation, a particular alcoxyamine of formula (A′), together with the use according to the invention of water-based dispersions containing the said polymers and possibly a polymer according to the prior art, in a process of manufacture of dry products containing mineral matter, as agents improving the opacity and whiteness of the said dry products.

In this example, the process of manufacture of the said dry products is characterised in that it includes a stage of grinding in water followed by a stage of concentration in water: both stages take place with the use of a polymer according to the prior art (case of prior art), or with use of a polymer according to the invention (case of the invention), or again with the use of a polymer according to the invention during the grinding stage and of a polymer according to the prior art during the concentration stage (case of the invention). The process leads to the obtaining of a water-based dispersion of mineral matter, enabling paper coatings to be manufactured.

The manufacturing process is also characterised in that it enables dry coverings, which are dry films resulting from the drying of the said paper coatings which have been previously applied on to papers, to be obtained.

### Preparation of Water-Based Dispersions of Mineral Matter.

In tests n^{o}29 to 32, one begins by grinding using a wet method, and using the methods well known to the skilled man in the art, a calcium carbonate sold by the company OMYA™ under the name Omyacarb™ 10 AV, by use of a polymer of the prior art or of a polymer according to the invention.

The solid content of the mineral matter suspension thus obtained, noted SC₁ (by percentage of dry weight of mineral matter relative to the total dry weight of the suspension), is then determined.

The percentage by weight of the particles the diameter of which is less than 1 μm and than 2 μm is then determined, noted %<1 μm and %<2 μm, using a Sedigraph™ 5100 device sold by the company MICROMERITICS™.

The suspension obtained is then concentrated in the presence of a dispersing agent of the prior art, or by use of a polymer according to.

When the grinding and concentration stages take place in the presence of polymers of the prior art, the dispersion obtained is characteristic of the prior art.

Conversely, when a polymer according to the invention is used during both stages or, when a polymer according to the invention is used during the grinding stage and a polymer of the prior art during the concentration stage, a dispersion according to the invention is obtained.

A dispersion of mineral matter is then obtained, the solid content of which is then measured, noted SC₂ (by dry weight of mineral matter relative to the total weight of the suspension), the granulometric characteristics of the latter as determined above remaining unchanged.

### Preparation of Paper Coatings and of Coated Papers.

2 types of paper coating are then produced, which enable 2 different types of paper to be coated.

In a first case, the dispersion thus obtained is then mixed with a styrene-acrylic binder sold under the name Acronal™ S360D by the company BASF™, in a proportion of 100 grams by dry weight of calcium carbonate for 12 parts of binder in its condition. With the preparation thus obtained, a polypropylene-based plastic paper sold under the name Synteape™ by the company ARJO-WIGGINS™ is thus coated using a laboratory coating machine of the Hand Coater™ type, model KC202, to obtain different coating weights between 5 and 50 g/m².

The samples are dried for 10 minutes at 90° C.

In a second case, a paper coating is produced containing:
68% by dry weight of mineral matter, relative to the total weight of the paper coating, introduced through the adequate quantity of dispersion of mineral matter previously prepared;
0.4% by dry weight relative to the dry weight of mineral matter, of Finnfix™ 10, which is a cellulose carboxy ethyl sold by the company METSA SERLA™;
0.4% by dry weight relative to the dry weight of mineral matter, of Mowiol™ 6.98, which is a polyvinylic alcohol sold by the company CLARIANT™;
11.0% by dry weight relative to the dry weight of mineral matter, of DL966, which is a latex sold by the company DOW™;
0.6% by dry weight relative to the dry weight of mineral matter, of Blancophor™ P, which is an optical brightener sold by the company CIBA™;

The pH of the paper coating is adjusted to 8.5 through the addition of sodium hydroxide. With the paper coatings obtained, pre-coated support papers, the grammage of which is equal to 76 g/m², are then coated, using a laboratory coating machine of the Hand Coater™ type, model KC202, in order to obtain a coat weight equal to 10 g/m².

The samples are dried for 10 minutes in an oven at 90° C.

The coated paper is then calendered at 80° C. under 40 bar pressure, using a calendering device of type RK 22 HU sold by the company DR DRAMISCH™ & CO.

Measurement of the Optical Properties of the Dry Films Resulting from the Drying of the Paper Coatings Applied on to the Papers.

The following are then determined:

in the case of coated and non-calendered plastic papers, as defined above:
the value of the light diffusion factor S (m²/kg), according to the method described in example 2;
   in the case of support papers which are pre-coated, and then coated and calendered, as defined above:
the value of the whiteness WCIE after calendering and according to norm ISO/FDIS 11475;
the value of the ratio R₂/R₃ after calendering, the parameters R₂ and R₃ being defined below:

Each coated sheet of paper is subjected to a light radiation of wavelength equal to 457 nm using an Elrepho™ 3000 spectrophotometer sold by the company DATACOLOR™ on a black plate to determine reflectance factor of the coated paper on a black background R₂ and on a pile of at least 10 coated sheets of paper to determine the reflectance factor R₃ of an opaque thickness of coated papers.

### Test N^{o}29

This test illustrates the prior art and uses during the grinding stage 0.50% by dry weight, relative to the dry weight of mineral matter, of a polymer of the prior art which is obtained by traditional radical polymerisation, and which consists, in terms of percentage by weight, of 81.5% methoxy polyethylene glycol methacrylate of molecular weight 2000, 4.9% methacrylic acid, and 13.6% acrylic acid, and 0.50% by dry weight of the same polymer relative to the dry weight of mineral matter during the concentration stage.

### Test N^{o}30

This test illustrates the invention and uses 0.50% by dry weight of polymer according to test n^{o}6 relative to the dry weight of mineral matter, during the grinding stage, and 0.50% by dry weight of the same polymer relative to the dry weight of mineral matter during the concentration stage.

### Test N^{o}31

This test illustrates the invention and uses 0.50% by dry weight of the polymer according to test n^{o}2, relative to the dry weight of mineral matter, during the grinding stage, and 0.70% by dry weight relative to the dry weight of mineral matter, of a polymer of the prior art which is obtained by traditional radical polymerisation, and which consists, in terms of percentage by weight, of 81.5% methoxy polyethylene glycol methacrylate of molecular weight 2000, 4.9% methacrylic acid, and 13.6% acrylic acid during the concentration stage.

### Test N^{o}32

This test illustrates the invention and uses 0.50% by dry weight of polymer according to test n^{o}3 relative to the dry weight of mineral matter, during the grinding stage, and 0.50% by dry weight of the same polymer relative to the dry weight of mineral matter during the concentration stage.

For tests n^{o}29 to 32, table 5 indicates:
the value of the solid content noted SC₁ (as a % by dry weight of mineral matter relative to the total dry weight of the suspension in question) for each suspension obtained after the grinding stage;
the value of the solid content noted SC₂ (as a % by dry weight of mineral matter relative to the total dry weight of the dispersion in question) for each dispersion obtained after the concentration stage;
the percentage by weight of particles the diameter of which is less than 1 μm and than 2 μm for each dispersion, respectively noted %<1 μm and %<2 μm;
measurements of the light diffusion factor S (m²/kg) for each coated plastic paper;
measurements of the ratio R₂/R₃ for pre-coated support papers which are then coated and calendered;
measurements of the whiteness WCIE for pre-coated support papers which are then coated and calendered;

TABLE 5
characteristics of the dispersions and of the water-based suspensions
of mineral matter and optical properties of the dry coverings.

| | | Characteristics of the dispersions | | | | Optical properties of | | |
|---|---|---|---|---|---|---|---|---|
| | | and of the suspensions of mineral | | | | the dry coverings | | |
| | | matter | | | | on plastic | on pre-coated | |
| Test | Prior art/ | SC₁ | SC₂ | | | papers | support papers | |
| n° | Invention | (%) | (%) | % <2 μm | % <1 μm | S (m²/kg) | R₂/R₃ | WCIE |
| 29 | Prior art | 38 | 69.8 | 96.9 | 69.7 | 131 | 95 | 115 |
| 30 | Invention | 38 | 70.5 | 97.1 | 72.8 | 138 | 95 | 118 |
| 31 | Invention | 38 | 70.6 | 96.6 | 69.7 | 143 | 95 | 120 |
| 32 | Invention | 38 | 70.1 | 95.9 | 69.7 | 140 | 95 | 119 |

A reading of table 5 demonstrates that the characteristics of the water-based dispersions according to the invention are similar to those of the dispersion of the prior art, in terms of solid content SC₂ and granulometry.

Conversely, in respect of the dry coverings resulting from the drying of the paper coatings on the plastic papers, their opacity, in terms of the light diffusion factor S, is substantially higher in the invention.

Similarly, with regard to the dry coverings resulting from the drying of the paper coatings on the pre-coated support papers, their opacity, in terms of measurement of the ratio R₂/R₃, is higher in the invention; the same goes for their whiteness.

This example thus demonstrates that the use according to the invention of the said polymers, together with the use according to the invention of the said dispersions containing the said polymers, enable the opacity and the whiteness of the dry coverings which are dry films resulting from the drying of paper coatings containing the polymers and the dispersions according to the invention to be improved substantially.

### EXAMPLE 7

This example illustrates the use according to the invention of controlled structure hydrosoluble polymers obtained by a process of controlled radical polymerisation using, as an initiator of polymerisation, a particular alcoxyamine of formula (A′), together with the use of the water-based suspensions containing the said polymers, in a process of manufacture of dry products containing mineral matter, as agents improving the opacity, brightness and whiteness of the said products.

In this example, the process of manufacture of the said products is characterised in that it includes a stage of grinding with use of a polymer according to the prior art or with use of a polymer according to the invention. The process thus leads to the obtaining of water-based suspensions of mineral matter. The said suspensions then enable paper coatings to be manufactured.

The manufacturing process is also characterised in that it leads dry coverings to be obtained which are dry films resulting from the drying of the said paper coatings which have been previously applied on to papers.

### Preparation of Water-Based Dispersions of Mineral Matter.

In tests n^{o}33 and 34, one begins by grinding using a wet method, and using the methods well known to the skilled man in the art, a calcium carbonate which is a calcite from Orgon (France), by use of a polymer of the prior art or of a polymer according to the invention.

The percentage by weight of the particles the diameter of which is less than 1 μm and than 2 μm is then determined, noted respectively %<1 μm and %<2 μm, using a Sedigraph™ 5100 device sold by the company MICROMERITICS™.

For each suspension its solid content SC, expressed as a percentage by dry weight of mineral matter relative to the total dry weight of the said suspension, is then determined.

### Preparation of Paper Coatings and of Coated Papers.

2 types of paper coating are then produced, which enable 2 different types of paper to be coated.

In a first case, the suspension thus obtained is then mixed with a styrene-acrylic binder sold under the name Acronal™ S360D by the company BASF™, in a proportion of 100 grams by dry weight of calcium carbonate for 12 parts of binder in its condition.

With the preparation thus obtained, a polypropylene-based plastic paper sold under the name Synteape™ by the company ARJO-WIGGINS™ is thus coated using a laboratory coating machine of the Hand Coater™ type, model KC202, to obtain different coating weights between 5 and 50 g/m₂.

The samples are then dried for 10 minutes at 90° C. in an oven.

The coated papers are then calendered at 90° C. under 60 bar pressure, using a calendering device of type RK 22 HU sold by the company DR DRAMISCH™ & CO.

In a second case, a paper coating is produced containing:
68% by dry weight of mineral matter, relative to the total weight of the paper coating, introduced through the adequate quantity of suspension of mineral matter previously prepared;
0.4% by dry weight relative to the dry weight of mineral matter, of Finnfix™ 10, which is a cellulose carboxymethyl sold by the company METSA SERLA™;
0.4% by dry weight relative to the dry weight of mineral matter, of Mowiol™ 6.98, which is a polyvinylic alcohol sold by the company CLARIANT™;
11.0% by dry weight relative to the dry weight of mineral matter, of DL966, which is a latex sold by the company DOW™;
0.6% by dry weight relative to the dry weight of mineral matter, of Blancophor™ P, which is an optical brightener sold by the company CIBA™;

The pH of the paper coating is adjusted to 8.5 through the addition of sodium hydroxide. With the paper coatings obtained, pre-coated support papers, the grammage of which is equal to 76 g/m², are then coated, using a laboratory coating machine of the Hand Coater™ type, model KC202, in order to obtain a coat weight equal to 10 g/m².

The samples are then dried for 10 minutes at 90° C. in an oven.

The coated paper is then calendered at 80° C. under 40 bar pressure, using a calendering device of type RK 22 HU sold by the company DR DRAMISCH™ & CO.

Measurement of Optical Properties in Relation to the Dry Films Resulting from the Drying of the Paper Coatings Applied on to the Papers.

The following are then determined:

in the case of coated plastic papers, as defined above:
the value of the light diffusion factor before calendering and according to the previously described method, noted S (m²/kg);
together with the value of the 75° TAPPI brightness after calendering and according to the method previously described, noted B_{1 75};
   in the case of support papers which are pre-coated, and then coated and calendered, as defined above:
the value of the ratio R₂/R₃ measured after calendering and according to the method described in example 6;
the value of the 75° TAPPI brightness after calendering and according to the method previously described, noted B_{2 75};
and the value of the whiteness after calendering and according to the method previously described, noted WCIE.

### Test N^{o}33

This test illustrates the prior art and uses during the grinding stage 0.65% by dry weight, relative to the dry weight of mineral matter, of a polymer of the prior art, which is a homopolymer of acrylic acid neutralised by a mixture of 30% by weight of calcium hydroxide and 70% by weight of sodium hydroxide (in molar equivalence).

### Test N^{o}34

This test illustrates the invention and uses during the grinding stage 11.35% by dry weight, relative to the dry weight of mineral matter, of the polymer obtained during test n^{o}12.

For tests n^{o}33 and 34, table 6 indicates:
the value of the solid content noted SC (as a % by dry weight of mineral matter relative to the total dry weight of the dispersion in question) for each dispersion obtained after the grinding stage;
the percentage by weight of particles the diameter of which is less than 1 in and than 2 μm for each suspension, respectively noted %<1 μm and %<2 μm;
for the coated plastic papers as defined above, the value of the light diffusion factor before calendering, noted S (m²/kg), together with the 75° TAPPI brightness after calendering, noted B_{1 75};
for the support papers which have been pre-coated, and then coated as defined above, and calendered as defined above, the value of the ratio R₂/R₃ measured after calendering, the value of the 75° TAPPI brightness after calendering, noted B_{2 75} and the value of the whiteness after calendering, noted WCIE.

TABLE 6
characteristics of the water-based suspensions of mineral
matter and optical properties of the dry coverings.

| | | | | | Properties of | | | | |
|---|---|---|---|---|---|---|---|---|---|
| | | Characteristics of the | | | the dry coverings | | | | |
| | | suspensions of mineral | | | on plastic | | on pre-coated | | |
| Test | Prior art/ | matter | | | papers | | support papers | | |
| n° | Invention | SC (%) | % <1 μm | % <2 μm | S (m²/kg) | B_{1 75} | R₂/R₃ | B_{2 75} | WCIE |
| 33 | Prior art | 74.5 | 77.8 | 98.9 | 97.4 | 69.2 | 91.1 | 66.0 | 122 |
| 34 | Invention | 73.9 | 77.5 | 99.0 | 110.7 | 72.4 | 91.7 | 69.0 | 125 |

A reading of table 6 demonstrates that the characteristics of the water-based suspension according to the invention are similar to those of the suspension according to the prior art, in ter s of solid content SC and granulometry.

Conversely, in respect of the dry coverings resulting from the drying of the paper coatings on the plastic papers, their opacity, in terms of measurement of the light diffusion factor S, and their brightness, in terms of measurement of the factor B_{1 75}, are better in the invention.

Similarly, in respect of the dry coverings resulting from the drying of the paper coatings on the pre-coated support papers, the opacity, in terms of measurement of the ratio R₂/R₃, their brightness, in terms of the measurement of factor B_{1 75}, and also their whiteness, in terms of measurement of the WCIE factor, are better in the invention.

This example thus demonstrates that the use according to the invention of the said polymers, together with the use according to the invention of the said suspensions containing the said polymers, enable the opacity, brightness and whiteness of the dry coverings which are dry films resulting from the drying of paper coatings containing the said polymers and the said suspensions to be improved substantially.

### EXAMPLE 8

This example illustrates the use according to the invention of controlled structure hydrosoluble polymers obtained by a process of controlled radical polymerisation using, as an initiator of polymerisation, a particular alcoxyamine of general formula (A′), in a process of manufacture of dry products containing mineral matter, as agents improving the brightness of the said products.

In this example, the process of manufacture of water-based formulations of mineral matter is characterised in that the said polymers are used as direct additives in the manufacture of water-based paints, which enable, after drying, dry paint films to be obtained.

For tests n^{o}35 to 38, different gloss paint formulations are produced, the compositions of which are given in table 7.

TABLE 7
composition (masses in grams of constituent elements) of the gloss
paints formulated by use of a polymer according to the prior art
and by direct use of polymers according to the invention.

| | Test n°35 | Test n°36 | Test n°37 | Test n°38 |
|---|---|---|---|---|
| Constituent elements | Prior art | Invention | Invention | Invention |
| propylene glycol | 25.0 | 25.0 | 25.0 | 25.0 |
| water | 59.5 | 58.0 | 58.7 | 58.8 |
| Coatex ™ BR3 | 5.0 | 0.0 | 0.0 | 0.0 |
| (40.0% volume by dry | | | | |
| weight of polymer) | | | | |
| Polymer according to | 0.0 | 6.5 | 0.0 | 0.0 |
| test n°4 | | | | |
| (30.6% solution by dry | | | | |
| weight of polymer) | | | | |
| polymer according to | 0.0 | 0.0 | 5.8 | 0.0 |
| test n°9 | | | | |
| (34.7% solution by dry | | | | |
| weight of polymer) | | | | |
| polymer according to | 0.0 | 0.0 | 0.0 | 5.7 |
| test n°10 | | | | |
| (35.2% solution by dry | | | | |
| weight of polymer) | | | | |
| Mergal ™ K6N | 1.5 | 1.5 | 1.5 | 1.5 |
| Byk ™ 34 | 2 | 2 | 2 | 2 |
| TiO2 RHD2 | 230.0 | 230.0 | 230.0 | 230.0 |
| Neocryl ™ XK 76 | 580.0 | 580.0 | 580.0 | 580.0 |
| Ammonia (31% solution | 7.0 | 7.0 | 7.0 | 7.0 |
| by weight) | | | | |
| Ethyl diglycol | 25.0 | 25.0 | 25.0 | 25.0 |
| Butyl diglycol | 25.0 | 25.0 | 25.0 | 25.0 |
| Coatex ™ Rheo 2000 ™ | 40.0 | 40.0 | 40.0 | 40.0 |
| Total | 1000.0 | 1000.0 | 1000.0 | 1000.0 |

Mergal ™ K6N is a bactericide sold by the company TROY ™
Byk ™ 34 is an anti-foaming agent sold by the company BYK ™.
TiO2 RHD2 is a titanium dioxide powder sold by the company HUNTSMAN ™
Neocryl ™ XK 76 is an acrylic binder sold by the company DSM ™
Coatex ™ Rheo 2000 ™ is an associative acrylic thickening agent sold by the company
COATEX ™

The formulations corresponding to tests n^{o}35, 36, 37 and 38 thus contain 0.2% by dry weight, relative to the total weight of the formulation, respectively of Coatex™ BR3, of the polymer according to test n^{o}4, of the polymer according to test n^{o}9, and of the polymer according to test n^{o}10.

Each paint was then applied using a standard manual film-stretcher, in a thickness of 150 μm on a glass plate, and then dried for 72 hours under the standard conditions well known to the skilled man in the art.

The brightness of the dry films obtained was measured on a Micro-Tri-Gloss™ reflectometer sold by the company BYK GARDNER™ at angles equal to 20°, 60° and 85° (BS20, BS60 and BS85) according to norm NF T 30-064. The corresponding results are shown in table 8.

TABLE 8
measurement of brightnesses at angles equal to 20°, 60° and
85° (BS20, BS60 and BS85) according to norm NF T 30-064.

| | Test n°35 | Test n°36 | Test n°37 | Test n°38 |
|---|---|---|---|---|
| | Prior art | Invention | Invention | Invention |
| BS20 | 52 | 56 | 54 | 54 |
| BS60 | 77 | 79 | 79 | 79 |
| BS85 | 94 | 96 | 96 | 96 |

A reading of table 8 demonstrates that the brightness is improved at all 3 angles, by direct use of the polymers according to the invention.

The direct use of the polymers according to the invention thus enables the brightness of the dry coverings which are dry films resulting from the drying of a water-based paint formulation containing the said polymers to be improved substantially.

### EXAMPLE 9

This example illustrates the use according to the invention of controlled structure hydrosoluble polymers obtained by a process of controlled radical polymerisation using, as an initiator of polymerisation, a particular alcoxyamine of formula (A′), together with the use according to the invention of water-based dispersions of mineral matter containing the said polymers, in a process of manufacture of dry products containing mineral matter, as agents improving the brightness of the said dry products.

In this example, the process of manufacture of the said dry products is characterised in that it includes a stage of grinding in water with the use of the said polymers, leading to the obtaining of a water-based suspension of mineral matter containing the said polymers. The said suspension is then dried so as to obtain a dry powder of mineral matter according to the invention. The said powder then allows the manufacture of plastics the brightness of which is improved relative to the plastics of the prior art.

### Preparation of a Calcium Carbonate Suspension Ground According to the Invention.

One begins by grinding in water a calcium carbonate which is a calcite from Orgon, in the presence of 0.70% by dry weight relative to the dry weight of calcium carbonate, of the polymer according to test n^{o}12.

A suspension of refined calcium carbonate is then obtained, the content by weight of which is equal to 60% by dry weight of calcium carbonate relative to the total mass of the suspension, and the percentages by weight of particles, the diameter of which is less than 1 μm and than 2 μm, and measured using a Sedigraph™ 5100 device, are respectively equal to 57.6% and 93.0%.

### Drying of the Suspension According to the Invention.

The previously obtained suspension is then dried in a Niro™ MINOR MOBIL 2000 atomiser, applying the following parameters: entry temperature equal to 350° C., removal temperature between 102 and 105° C., ventilation adjusted to 99%, air pressure equal to 4 bar.

2 drying operations are then undertaken, one by adding during drying 0.8% by dry weight of stearic acid relative to the dry weight of calcium carbonate (test n^{o}41), and the other without addition of stearic acid (test n^{o}40).

The two dry powders then obtained, corresponding to tests n^{o}41 and n^{o}42, were evaluated in a rigid PVC formulation, of the window profile type, comparatively with a product of the prior art which is Omyacarb™ 95 T, sold by the company OMYA™, and which corresponds to test n^{o}39.

### Production of Dry-Blends.

A formulation known as a “dry-blend” according to the name well known to the skilled man in the art is firstly produced, by hot mixing, in a 5-litre fast mixer of the Guédu™ type, of the following constituents:

| | | |
|---|---|---|
| PVC Lacovyl ™ S110P resin (ARKEMA ™) | 100 | parts |
| Baroban ™ stabiliser (BARLOCHER ™) | 2.7 | parts |
| Lacowax ™ EP lubricant (CLARIANT ™) | 0.05 | parts |
| dry calcium carbonate powder | 15 | parts |

Tests n^{o}41 and n^{o}40 correspond to the dry-blends using the dry calcium carbonate powder according to the invention, respectively with or without addition of stearic acid, and test n^{o}39 corresponds to the prior art using the product Omyacarb™ 95 T.

The mixing cycle in the Guédu mixer is as follows:
pre-heating of the mixer for 30 minutes at 50° C.;
introduction of the PVC resin and increase of the temperature to 90° C.;
addition of the stabilising agent and of the lubricant;
increase of the temperature to 115° C. and introduction of the dry calcium carbonate powder;
stirring for 15 minutes, followed by discharge of the mixture and cooling of the dry-blend.

Extrusion of Profiles.

These 3 dry-blends were extruded using the POLYLAB™ device sold by the company THERMOELECTRON™ equipped with a standard conical twin screw (ref: 557-2210) and with the sheet die.

The temperature of the extrusion device is set at 180° C. and the extrusion speed at 30 rpm. For each of the tests n^{o}39 and n^{o}41, the values of the motor torque, the pressure in the screw head and the flow rate were noted.

TABLE 9
values of the motor torque, the pressure in the screw head
and the flow rate, measured during extrusion of the dry-blends
corresponding to tests n°39, n°40 and n°41.

| | | | linear |
|---|---|---|---|
| | Torque | Pressure | speed |
| Test n° | (Nm) | (bar) | (cm/min.) |
| 39 | 100 | 110 | 67.0 |
| 40 | 120 | 125 | 67.0 |
| 41 | 100 | 120 | 69.5 |

No formation of any agglomerate is observed during the tests.

Furthermore, in the case of the tests corresponding to the invention, a very satisfactory dispersion of the load within the polymer matrix is observed, which reflects a satisfactory compatibility of the dry powders according to the invention with the resin.

### Brightness Measurements.

The brightness measurements were made using a Micro-Tri-Gloss™ reflectometer sold by the company BYK GARDNER™, at an angle of exposure equal to 85°.

The values obtained are the result of the arithmetic average made over 5 measurements, and are indicated in table 10.

TABLE 10
measurement of brightness at 85° for the extruded products
corresponding to tests n°39, n°40 and n°41.

| | Brightness |
|---|---|
| Test n° | measured at 85° |
| 39 | 68 |
| 40 | 71 |
| 41 | 78 |

The results of table 10 demonstrate that the brightness is improved by direct use of the polymers according to the invention.

Direct use of the polymers according to the invention thus enables the brightness of the dry products which are plastics containing the said polymers to be improved.

### EXAMPLE 10

This example illustrates the process whereby hydrosoluble polymers are obtained, use of which in the subsequent examples forms the object of the invention.

In tests n^{o}42 to 47, a process of controlled radical polymerisation is implemented using methods well known to the skilled man in the art, using as an initiator of polymerisation alcoxyamine of formula (A′).

Controlled structure hydrosoluble polymers are then obtained, the monomer composition and molecular weight of which (measured according to the method previously described and expressed in g/mole) are given in tests n^{o}42 to 47.

### Test N^{o}42

This test leads to the obtaining, using a process of controlled radical polymerisation using as an initiator of polymerisation alcoxyamine of formula (A′), a hydrosoluble polymer of composition by weight of, relative to the total mass of monomers used:
18.2% methacrylic acid,
78.9% ethylene glycol methacrylate,
2.9% butoxypolyoxypropylene hemimaleate containing 19 units of oxypropylene,
   of which the previously described GPC analysis indicates to us an average molecular mass by weight Mw of 25,170 g/mole.

This GPC method also enables the polymolecularity index of this product to be determined, which is equal to 3.35.

### Test N^{o}43

This test leads to the obtaining, using a process of controlled radical polymerisation using as an initiator of polymerisation alcoxyamine of formula (A′), a hydrosoluble polymer of composition by weight of, relative to the total mass of monomers used:
60.0% methacrylic acid,
37.1% methoxy polyethylene glycol methacrylate of molecular mass 2000,
2.9% butoxypolyoxypropylene hemimaleate containing 19 units of oxypropylene,
   of which the previously described GPC analysis indicates to us an average molecular mass by weight Mw of 25,970 g/mole.

This GPC method also enables the polymolecularity index of this product to be determined, which is equal to 2.0.

### Test N^{o}44

This test leads to the obtaining, using a process of controlled radical polymerisation using as an initiator of polymerisation alcoxyamine of formula (A′), a hydrosoluble polymer of composition by weight of, relative to the total mass of monomers used:
80.0% methacrylic acid,
17.1% methoxy polyethylene glycol methacrylate of molecular mass 2000,
2.9% butoxypolyoxypropylene hemimaleate containing 19 units of oxypropylene,
   of which the previously described GPC analysis indicates to us an average molecular mass by weight Mw of 25,970 g/mole.

This GPC method also enables the polymolecularity index of this product to be determined, which is equal to 1.8.

### Test N^{o}45

This test leads to the obtaining, using a process of controlled radical polymerisation using as an initiator of polymerisation alcoxyamine of formula (A′), a hydrosoluble polymer of composition by weight of, relative to the total mass of monomers used:
4.8% methacrylic acid,
78.9% methoxy polyethylene glycol methacrylate of molecular mass 2000,
13.4% ethylene glycol methacrylate phosphate,
2.9% butoxypolyoxypropylene hemimaleate containing 19 units of oxypropylene,
   of which the previously described GPC analysis indicates to us an average molecular mass by weight Mw of 63,930 g/mole.

This GPC method also enables the polymolecularity index of this product to be determined, which is equal to 2.69.

### Test N^{o}46

This test leads to the obtaining, using a process of controlled radical polymerisation using as an initiator of polymerisation alcoxyamine of formula (A′), a hydrosoluble polymer of composition by weight of, relative to the total mass of monomers used:
18.2% methacrylic acid,
78.9% methoxy polyethylene glycol methacrylate of molecular mass 2000,
2.9% of a sililated monomer of formula R-A-Si (OB)₃, where R designates the methacrylate group, A designates the propyl radical and B designates the methyl radical,
   of which the previously described GPC analysis indicates to us an average molecular mass by weight Mw of 48,330 g/mole.

This GPC method also enables the polymolecularity index of this product to be determined, which is equal to 3.84.

### Test N^{o}47

This test leads to the obtaining, using a process of controlled radical polymerisation using as an initiator of polymerisation alcoxyamine of formula (A′), a hydrosoluble polymer of composition by weight of, relative to the total mass of monomers used:
8.1% methacrylic acid,
69.9% methoxy polyethylene glycol methacrylate of molecular mass 2000,
19.4% of [2-(methacryloyloxy)ethyl]trimethyl ammonium chloride,
2.9% butoxypolyoxypropylene hemimaleate containing 19 units of oxypropylene,
   the GPC analysis of which has not been undertaken.

### EXAMPLE 11

This example illustrates the use according to the invention of controlled structure hydrosoluble polymers obtained by a process of controlled radical polymerisation using, as an initiator of polymerisation, a particular alcoxyamine of formula (A′), together with the use of the water-based suspensions containing the said polymers, in a process of manufacture of dry products containing mineral matter, as agents improving the opacity and brightness of the said products.

In this example, the process of manufacture of the said products is characterised in that it includes a stage of grinding with use of a polymer according to the prior art or with use of a polymer according to the invention. The process thus leads to the obtaining of water-based suspensions of mineral matter. The said suspensions then enable paper coatings to be manufactured.

The manufacturing process is also characterised in that it leads dry coverings to be obtained which are dry films resulting from the drying of the said paper coatings which have been previously applied on to papers.

### Preparation of Water-Based Dispersions of Mineral Matter.

In tests n^{o}48 to 52, one begins by grinding using a wet method, and using the methods well known to the skilled man in the art, a calcium carbonate which is a calcite from Orgon (France), by use of a polymer of the prior art or of a polymer according to the invention.

The percentage by weight of the particles the diameter of which is less than 1 Um and than 2 μm is then determined, noted respectively %<1 μm and %<2 μm, using a Sedigraph™ 5100 device sold by the company MICROMERITICS™.

For each suspension its solid content SC, expressed as a percentage by dry weight of mineral matter relative to the total weight of the said suspension, is then determined.

### Preparation of Paper Coatings and of Coated Papers.

For tests no 48 to 52, the suspension thus obtained is then mixed with a styrene-acrylic binder sold under the name Acronal™ S360D by the company BASF™, in a proportion of 100 grams by dry weight of calcium carbonate for 12 parts of binder in its condition.

With the preparation thus obtained, a polypropylene-based plastic paper sold under the name Synteape™ by the company ARJO-WIGGNS™ is thus coated using a laboratory coating machine of the Hand Coater™ type, model KC202, to obtain different coating weights between 5 and 50 g/m².

The samples are then dried for 10 minutes at 90° C. in an oven.

The coated papers are then calendered at 90° C. under 60 bar pressure, using a calendering device of type RK 22 HU sold by the company DR DRAMISCH™ & CO.

Measurement of Optical Properties in Relation to the Dry Films Resulting from the Drying of the Paper Coatings Applied on to the Papers.

The following are then determined:

in the case of coated plastic papers, as defined above:
the value of the light diffusion factor before calendering and according to the previously described method, noted S (m²/kg);
together with the value of the 75° TAPPI brightness after calendering and according to the method previously described, noted B₇₅;

Test no 48 illustrates the prior art and uses during the grinding stage 0.65% by dry weight, relative to the dry weight of mineral matter, of a polymer of the prior art, which is a homopolymer of acrylic acid neutralised by a mixture of 50% by weight of sodium hydroxide and 50% by weight of magnesium hydroxide (in molar equivalence).

Tests no 49 to 52 illustrate the invention and use during the grinding stage 1.2% by dry weight, relative to the dry weight of mineral matter, of a polymer which is respectively the polymer described in tests no 43, 44, 45 and 46.

TABLE 11
characteristics of the water-based suspensions of mineral matter
and optical properties of the dry coverings (* not measured).

| | | Characteristics of the | | | | |
|---|---|---|---|---|---|---|
| | | suspensions of mineral | | | | |
| | | matter | | | Properties of | |
| Test | Prior art/ | ES | | | the dry coverings | |
| n° | Invention | (%) | % <1 μm | % <2 μm | S (m²/kg) | B₇₅ |
| 48 | Prior art | 77.9 | 59.6 | 88.1 | 87 | 68 |
| 49 | Invention | 78.2 | 62.4 | 90.2 | 111 | 73 |
| 50 | Invention | 78.0 | 62.1 | 89.2 | * | * |
| 51 | Invention | 77.8 | 60.1 | 91.2 | 94 | 69 |
| 52 | Invention | 78.3 | 53.4 | 90.8 | 114 | 72 |

A reading of table 11 demonstrates that the characteristics of the water-based suspension according to the invention are similar to those of the suspension according to the prior art, in terms of solid content SC and granulometry.

Conversely, in respect of the dry coverings resulting from the drying of the paper coatings on the plastic papers, their opacity, in terms of measurement of the light diffusion factor S, and their brightness, in terms of measurement of the factor B 75, are better in the invention.

This example thus demonstrates that the use according to the invention of the said polymers, together with the use according to the invention of the said suspensions containing the said polymers, enable the opacity and brightness of dry coverings which are dry films resulting from the drying of paper coatings containing the said polymers and the said suspensions to be improved substantially.

In addition, the Applicant wishes to state that the water-based suspensions of mineral matter obtained for tests n^{o}49 to 52 contain no foam, unlike the suspension obtained for test n^{o}48. This is due to the use of the butoxypolyoxypropylene hemimaleate monomer containing 19 units of oxypropylene and of the sililated monomer of formula R-A-Si (OB)₃, where R designates the methacrylate group, A designates the propyl radical, and B designates the methyl radical, which have an antifoam character.

### EXAMPLE 12

This example illustrates the use according to the invention of controlled structure hydrosoluble polymers obtained by a process of controlled radical polymerisation using, as an initiator of polymerisation, a particular alcoxyamine of formula (A′), together with the use of the water-based suspensions containing the said polymers, in a process of manufacture of dry products containing mineral matter, as agents improving the brightness of the said products.

In this example, the process of manufacture of the said products is characterised in that it includes a stage of grinding with use of a polymer according to the prior art or with use of a polymer according to the invention. The process thus leads to the obtaining of water-based suspensions of mineral matter. The said suspensions are dried and then enable paints to be manufactured.

The manufacturing process is also characterised in that it leads dry coverings to be obtained which are dry films resulting from the drying of the said paints which have been previously applied on to glass plates.

For tests n^{o}53 and 54, the first stage is to grind a calcium carbonate sold by the company OMYA™ under the name Omyacarb™ 400, using 0.4% by weight of a polymer according to the prior art which is:
an acrylic acid homopolymer neutralised by a mixture of 50% by weight of sodium hydroxide and 50% by weight of magnesium hydroxide (in molar equivalence) for test n^{o}53,
the polymer according to test n^{o}42 for test n^{o}54 which illustrates the invention.

The suspension obtained has a dry extract equal to 60%.

The latter is dried.

A paint is then produced the composition of which is as follows:
Synolac™ 6868 WL 75 sold by the company CRAY VALLEY™ (280 parts),
Super Gelkyd™ 3915 WDA 55 sold by the company CRAY VALLEY™ (40 parts),
White spirit (84 parts),
Kronos™ 2310 sold by the company KRONOS™ (220 parts),
350 parts of the dried suspension of calcium carbonate according to test 53 or 54,
Dry VP 013 sold by the company BORCHERS™ (11 parts),
Borchinox™ M2 sold by the company BORCHERS™ (3 parts),
Dowanol™ sold by the company DOW™ (12 parts).

Each paint (for test 53 and test 54) was then applied using a standard manual film-stretcher, in a thickness of 150 μm on a glass plate, and then dried for 72 hours under the standard conditions well known to the skilled man in the art.

The brightness of the dry films obtained was measured on a Micro-Tri-Gloss™ reflectometer sold by the company BYK GARDNER™ at an angle equal to 20° according to norm NF T 30-064.

For test n^{o}53, a brightness value equal to 58 is obtained, while it is equal to 70 for test n^{o}54: this result thus clearly demonstrates that the use of the polymer according to the invention enables the brightness of a dry covering which is a dry paint film to be increased.

### SUMMARY

The present invention concerns the sector of agents enabling the improvement of optical properties, such as notably opacity and/or brightness, of mineral matter-based dry products containing the said agents.

The invention concerns the use in a manufacturing process of mineral matter-based dry products, as agents improving opacity and/or brightness of the said dry products, of hydrosoluble polymers of controlled structure obtained by a process of controlled radical polymerisation using, as the polymerisation initiator, a particular alcoxyamine. The invention also concerns the use for the same purposes of dispersions and/or of water-based suspensions of mineral matter containing the said polymers.

The dry products concerned are either plastics, or filled papers, or dry films resulting from the drying of a paper coating, dry films resulting from the drying of a water-based paint formulation, or again dry films resulting from the drying of a water-based cosmetic formulation, containing mineral matter together with the said polymers.

1. A method of manufacturing a dry product comprising a mineral matter, said method comprises:
(i) preparing a water-based dispersion and/or suspension comprising the mineral matter and hydrosoluble polymers having a controlled structure and are obtained by a process of controlled radical polymerisation using as an initiator of polymerisation a particular alcoxyamine of general formula (A): 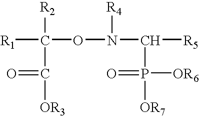
where:
R₁ and R₂ represent an alkyl radical, linear or branched, with a number of carbon atoms ranging from 1 to 5,
R₃ represents a hydrogen atom, an alkyl radical, linear or branched, with a number of carbon atoms ranging from 1 to 8, a phenyl radical, a cation such as Li+, Na+, K+, H4N+, Bu3HN⁺, where Bu=butyl,
R₄ represents an alkyl radical, linear or branched, with a number of carbon atoms ranging from 1 to 8,
R₅ represents an alkyl radical, linear or branched, with a number of carbon atoms ranging from 1 to 8,
R₆ and R₇ represent an alkyl radical, linear or branched, with a number of carbon atoms ranging from 1 to 8,
(ii) coating a material with the suspension, and
(iii) drying the coated material, thereby obtaining the dry product having the improved brightness ado oacity.

2. The method according to claim 1, comprising at least one of grinding, dispersing and concentrating the mineral matter in water, which leads to a dispersion and/or aqueous suspension of mineral matter being obtained.

3-4. (canceled)

5. The method according to claim 1 wherein the polymers are obtained by a process of polymerisation of monomers selected from the group consisting of:
a) at least one ionic monomer which is either:
i) anionic with a carboxylic or dicarboxylic or phosphoric or phosphonic or sulphonic function, or their mixtures, or
ii) cationic, or
iii) a mixture of i) and ii)
b) and at least one non-ionic Monomer, where the non-ionic Monomer consists of at least one monomer of formula (I): 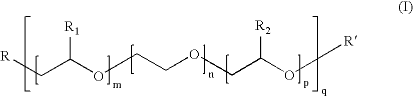
where:
m and p represent a number of alkylene oxide units of less than or equal to 150,
n represents a number of ethylene oxide units of less than or equal to 150,
q represents a whole number at least equal to 1 and such that 5≦(m+n+p)q≦
R₁ represents hydrogen or the methyl or ethyl radical,
R₂ represents hydrogen or the methyl or ethyl radical,
R represents a radical containing an unsaturated polymerisable function, belonging to the group of vinylics, or to the group of acrylic, methacrylic, maleic, itaconic, crotonic, vinylphthalic esters, or to the group of unsaturated urethanes selected from the group consisting of acrylurethane, methacrylurethane, α-α′ dimethyl-isopropenyl-benzylurethane, and allylurethane, or to the group of allylic or vinylic ethers, whether or not substituted, or to the group of ethylenically unsaturated amides or imides,
R′ represents hydrogen or a hydrocarbonate radical having 1 to 40 atoms of carbon,
or a mixture of several monomers of formula (I),
c) and possibly at least one monomer of the acrylamide or methacrylamide type and their mixtures, or at least one non-hydrosoluble monomer, the vinylics, or at least one organofluorate or organosililate monomer, or their mixtures,
d) and possibly at least one monomer having at least two ethylenic unsaturations, or of the mixture of several of these monomers.

6. The method according to claim 5, wherein the hydrosoluble polymers are obtained by controlled radical polymerisation of monomers selected from the group consisting of:
a) at least one ionic monomer which is either:
i) anionic with ethylenic unsaturation and with a monocarboxylic function in the acid or acid-salt state chosen from among the ethylenic unsaturation monomers and with a monocarboxylic function, or the diacid hemiesters such as the C₁ to C₄ monoesters of maleic or itaconic acids, or chosen from among the monomers with ethylenic unsaturation and with a dicarboxylic function in the acid or acid-salt state, or the anhydrides of carboxylic acids, or chosen from among the monomers with ethylenic unsaturation and with a sulphonic function in the acid or acid-salt state, or chosen from among the monomers with ethylenic unsaturation and with a phosphoric function in the acid or acid-salt state, or chosen from among the monomers with ethylenic unsaturation and with a phosphonic function in the acid or acid-salt state, or
ii) cationic chosen from among N-[3-(dimethylamino) propyl]acrylamide or N-[3-(dimethylamino) propyl]methacrylamide, the unsaturated esters such as N-[2-(dimethylamino) ethyl]methacrylate, or N-[2-(dimethylamino) ethyl]acrylate, or from among the quaternary ammonium compounds such as [2-(methacryloyloxy)ethyl]trimethyl ammonium chloride or sulphate, [2-(acryloyloxy)ethyl]trimethyl ammonium chloride or sulphate, [3-(acrylamido) propyl]trimethyl ammonium chloride or sulphate, dimethyl diallyl ammonium chloride or sulphate, [3-(methacrylamido) propyl]trimethyl ammonium chloride or sulphate, or their mixtures, or
iii) a mixture of the abovementioned anionic and cationic monomers
b) and a least one monomer with a non-ionic ethylenic unsaturation of formula (I): 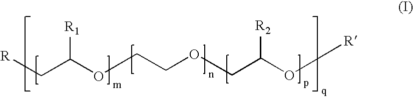
where:
m and p represent a number of alkylene oxide units of less than or equal to 150,
n represents a number of ethylene oxide units of less than or equal to 150,
q represents a whole number at least equal to 1 and such that 5≦(m+n+p)q≦150,
R₁ represents hydrogen or the methyl or ethyl radical,
R₂ represents hydrogen or the methyl or ethyl radical,
R represents a radical containing an unsaturated polymerisable function, belonging to the group of vinylics, or to the group of acrylic, methacrylic, maleic, itaconic, crotonic, vinylphthalic esters, or to the group of unsaturated urethanes, or to the group of allylic or vinylic ethers, whether or not substituted, or to the group of ethylenically unsaturated amides or imides,
R′ represents hydrogen or a hydrocarbonate radical having 1 to 40 atoms of carbon,
or a mixture of several monomers of formula (I),
c) and possibly at least one monomer of the acrylamide or methacrylamide type and their mixtures, or at least one non-hydrosoluble monomer, the vinylics, or at least one organofluorate or organosililate monomer chosen preferentially from among the molecules of formulae (IIa) or (IIb): 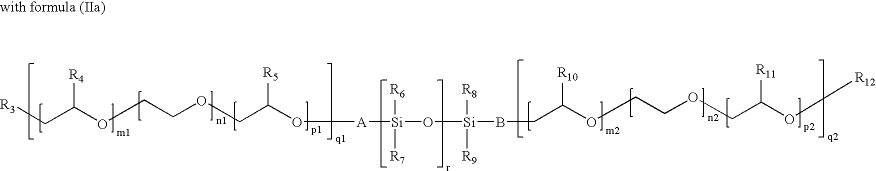
where:
m₁, p₁, m₂ and p₂ represent a number of alkylene oxide units of less than or equal to 150,
n₁ et n₂ represent a number of ethylene oxide units of less than or equal to 150,
q₁ and q₂ represent a whole number at least equal to 1 and such that 0≦(m₁+n₁+p₁)q₁≦150 and 0≦(m₂+n₂+p₂)q₂≦150,
r represents a number such that 1≦r≦200,
R₃ represents a radical containing an unsaturated polymerisable function, belonging to the group of vinylics, or to the group of acrylic, methacrylic, maleic, itaconic, crotonic, vinylphthalic esters, or to the group of unsaturated urethanes, or to the group of allylic or vinylic ethers, whether or not substituted, or to the group of ethylenically unsaturated amides or imides,
R₄, R₅, R₁₀ and R₁₁ represent hydrogen or the methyl or ethyl radical,
R₆, R₇, R₈ and R₉ represent linear or branched alkyl or aryl, or alkylaryl or arylalkyl groupings, having 1 to 20 carbon atoms, or their mixtures,
R₁₂ represents a hydrocarbonated radical having 1 to 40 carbon atoms,
A and B are groupings which may be present, which then represent a hydrocarbonated radical having 1 to 4 carbon atoms,
with formula (IIb)
R-A-Si(OB)₃
where:
R represents a radical containing an unsaturated polymerisable function, belonging to the group of vinylics, or to the group of acrylic, methacrylic, maleic, itaconic, crotonic, vinylphthalic esters, or to the group of unsaturated urethanes or to the group of allylic or vinylic ethers, whether or not substituted, or to the group of ethylenically unsaturated amides or imides,
A is a grouping which may be present, which then represents a hydrocarbonated radical having 1 to 4 carbon atoms,
B represents a hydrocarbonated radical having 1 to 4 carbon atoms, or the mixture of several of these monomers,
d) and possibly at least one crosslinking monomer chosen from the group constituted by ethylene glycol dimethacrylate, trinmethylolpropanetriacrylate, allyl acrylate, the allyl maleates, methylene-bis-acrylamide, methylene-bis-methacrylamide, tetrallyloxyethane, the triallylcyanurates, the allyl ethers obtained from polyols or chosen from among the molecules of formula (III): 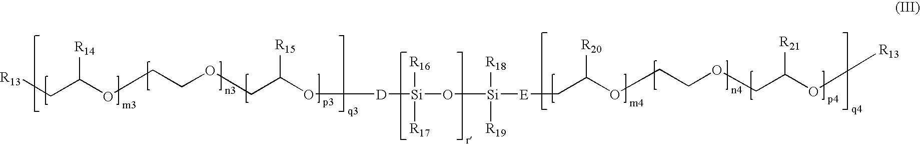
where:
m₃, p₃, m₄ and p₄ represent a number of alkylene oxide units of less than or equal to 150,
n₃ et n₄ represent a number of ethylene oxide units of less than or equal to 150,
q₃ and q₄ represent a whole number at least equal to 1 and such that 0≦(m₃+n₃+p₃)q₃≦150 and 0≦(m₄+n₄+p₄)q₄≦150,
r′ represents a number such that 1≦r′≦200,
R₁₃ represents a radical containing an unsaturated polymerisable function, belonging to the group of vinylics, or to the group of acrylic, methacrylic, maleic, itaconic, crotonic, vinylphthalic esters, or to the group of unsaturated urethanes, or to the group of allylic or vinylic ethers, whether or not substituted, or to the group of ethylenically unsaturated amides or imides,
R₁₄, R₁₅, R₂₀ and R₂₁ represent hydrogen or the methyl or ethyl radical,
R₁₆, R₁₇, R₁₈ and R₁₉ represent linear or branched alkyl or aryl, or alkylaryl or arylalkyl groupings, having 1 to 20 carbon atoms, or their mixtures,
D and E are groupings which may be present, which then represent a hydrocarbonated radical having 1 to 4 carbon atoms,
or the mixture of several of these monomers.

7. The method according to claim 1, wherein the said polymers are constituted, expressed as weight, relative to the total mass of monomers used:
a) from 2% to 98% of a least one ionic monomer which is either:
i) an anionic monomer with ethylenic unsaturation and with a monocarboxylic function in the acid or acid-salt state chosen from among the ethylenic unsaturation monomers and with a monocarboxylic function, or the diacid hemiesters such as the C₁ to C₄ monoesters of maleic or itaconic acids, or chosen from among the monomers with ethylenic unsaturation and with a dicarboxylic function in the acid or acid-salt state, or the anhydrides of carboxylic acids, or chosen from among the monomers with ethylenic unsaturation and with a sulphonic function in the acid or acid-salt state, or chosen from among the monomers with ethylenic unsaturation and with a phosphoric function in the acid or chosen from among the monomers with ethylenic unsaturation and with a phosphonic function in the acid or acid-salt state, or their mixtures, or
ii) a cationic monomer chosen from among N-[3-(dimethylamino) propyl]acrylamide or N-[3-(dimethylamino) propyl]methacrylamide, the unsaturated esters such as N-[2-(dimethylamino) ethyl]methacrylate, or N-[2-(dimethyl amino) ethyl]acrylate, or from among the quaternary ammonium compounds such as [2-(methacryloyloxy)ethyl]trimethyl ammonium chloride or sulphate, [2-(acryloyloxy)ethyl]trimethyl ammonium chloride or sulphate, [3-(acrylamido) propyl]trimethyl ammonium chloride or sulphate, dimethyl diallyl ammonium chloride or sulphate, [3-(methacrylamido) propyl]trimethyl ammonium chloride or sulphate, or their mixtures, or
iii) a mixture of the abovementioned anionic and cationic monomers,
b) from 2 to 98% of a least one monomer with non-ionic ethylenic unsaturation of formula (I): 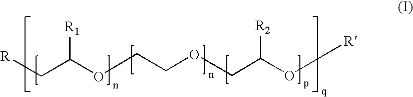
where:
m and p represent a number of alkylene oxide units of less than or equal to 150,
n represents a number of ethylene oxide units of less than or equal to 150,
q represents a whole number at least equal to 1 and such that 5≦(m+n+p)q≦150,
R₁ represents hydrogen or the methyl or ethyl radical,
R₂ represents hydrogen or the methyl or ethyl radical,
R represents a radical containing an unsaturated polymerisable function, belonging to the group of vinylics, or to the group of acrylic, methacrylic, maleic, itaconic, crotonic, vinylphthalic esters, or to the group of unsaturated urethanes, or to the group of allylic or vinylic ethers, whether or not substituted, or to the group of ethylenically unsaturated amides or imides,
R′ represents hydrogen or a hydrocarbonate radical having 1 to 40 atoms of carbon,
or a mixture of several monomers of formula (I),
c) from 0 to 50% of at least one monomer of the acrylamide or methacrylamide type and their mixtures, or again at least one non-hydrosoluble monomer such as the alkyl acrylates or methacrylates, the vinylics such as vinyl acetate, vinylpyrrolidone, styrene, alphamethylstyrene and their derivatives, or again at least one organofluorate or organosililate monomer selected from the molecules of formulae (IIa) and (IIb): 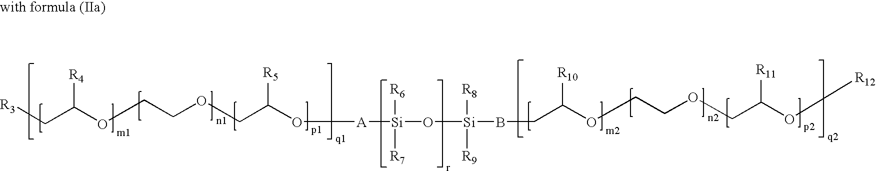
where:
m₁, p₁, m₂ and p₂ represent a number of alkylene oxide units of less than or equal to 150,
n₁ et n₂ represent a number of ethylene oxide units of less than or equal to 150,
q₁ and q₂ represent a whole number at least equal to 1 and such that 0≦(m₁+n₁+p₁)q₁≦150 and 0≦(m₂+n₂+p₂)q₂≦150,
r represents a number such that 1≦r≦200,
R₃ represents a radical containing an unsaturated polymerisable function, belonging to the group of vinylics, or to the group of acrylic, methacrylic, maleic, itaconic, crotonic, vinylphthalic esters, or to the group of unsaturated urethanes, or to the group of allylic or vinylic ethers, whether or not substituted, or to the group of ethylenically unsaturated amides or imides,
R₄, R₅, R₁₀ and R₁₁ represent hydrogen or the methyl or ethyl radical,
R₆, R₇, R₅ and R₉ represent linear or branched alkyl or aryl, or alkylaryl or arylalkyl groupings, having 1 to 20 carbon atoms, or their mixtures,
R₁₂ represents a hydrocarbonated radical having 1 to 40 carbon atoms,
A and B are groupings which may be present, which then represent a hydrocarbonated radical having 1 to 4 carbon atoms, with formula (IIb)
R-A-Si(OB)₃
where:
R represents a radical containing an unsaturated polymerisable function, belonging to the group of vinylics, or to the group of acrylic, methacrylic, maleic, itaconic, crotonic, vinylphthalic esters, or to the group of unsaturated urethanes, or to the group of allylic or vinylic ethers, whether or not substituted, or to the group of ethylenically unsaturated amides or imides,
A is a grouping which may be present, which then represents a hydrocarbonated radical having 1 to 4 carbon atoms,
B represents a hydrocarbonated radical having 1 to 4 carbon atoms, or the mixture of several of these monomers,
d) from 0 to 3% of at least one crosslinking monomer chosen from the group constituted by ethylene glycol dimethacrylate, trimethylolpropanetriacrylate, allyl acrylate, the allyl maleates, methylene-bis-acrylamide, methylene-bis-methacrylamide, tetrallyloxyethane, the triallylcyanurates, the allyl ethers obtained from polyols, or chosen from among the molecules of formula (III): 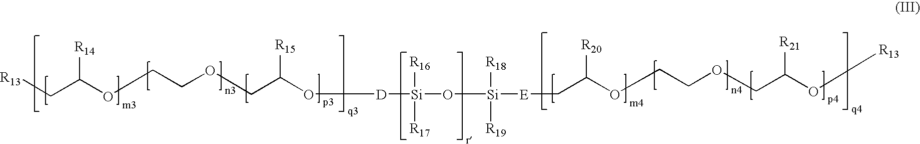
where:
m₃, p₃, m₄ and p₄ represent a number of alkylene oxide units of less than or equal to 150,
n₃ et n₄ represent a number of ethylene oxide units of less than or equal to 150,
q₃ and q₄ represent a whole number at least equal to I and such that 0≦(m₃+n₃+p₃)q₃≦150 and 0≦(m₄+n₄+p₄)q₄≦150,
r′ represents a number such that 1≦r′≦200,
R₁₃ represents a radical containing an unsaturated polymerisable function, belonging to the group of vinylics, or to the group of acrylic, methacrylic, maleic, itaconic, crotonic, vinylphthalic esters, or to the group of unsaturated urethanes, or to the group of allylic or vinylic ethers, whether or not substituted, or to the group of ethylenically unsaturated amides or imides,
R₁₄, R₁₅, R₂₀ and R₂₁ represent hydrogen or the methyl or ethyl radical,
R₁₆, R₁₇, R₁₈ and R₁₉ represent linear or branched alkyl or aryl, or alkylaryl or arylalkyl groupings, having 1 to 20 carbon atoms, or their mixtures,
D and E are groupings which may be present, which then represent a hydrocarbonated radical having 1 to 4 carbon atoms,
or the mixture of several of these monomers.

8. The method according to claim 1, wherein said polymers are obtained in the acid form and possibly distilled, and are partially or totally neutralised by one or more agents.

9. The method according to claim 1, wherein said polymers are, before or after the total or partial neutralisation reaction, treated and separated into several phases, by one or more polar solvents belonging to the group constituted by water, methanol, ethanol, propanol, isopropanol, the butanols, acetone, tetrahydrofuran, or their mixtures.

10. The method according to claim 1, wherein said polymers are dried.

11. The method according to claim 1, wherein said hydrosoluble polymers are hydrosoluble copolymers and have a structure of the statistic, block, comb, grafted or alternate type.

12. The method according claim 1, wherein the mineral matter is selected from the group consisting of a pigment and/or a mineral filler, chosen from among natural or synthetic calcium carbonate, the dolomites, kaolin, talc, cement, gypsum, lime, magnesia, titanium dioxide, satin white, aluminium trioxide, or aluminium trihydroxide, the silicas, mica and a mixture of these fillers one with another, or mixtures of calcium carbonate with aluminium trihydroxide or aluminium trioxide, or again mixtures with synthetic or natural fibres, or mineral costructures or their mixtures.

13. The method according to claim 12, wherein the mineral matter is natural or synthetic calcium carbonate.

14-24. (canceled)

25. A water-based dispersion of a mineral matter, comprising:
water,
at least one mineral matter,
at least one hydrosoluble polymer obtained by a process of controlled radical polymerisation using an alcoxyamine of formula (A),
and possibly at least one dispersing agent different from the polymer.

26. The water-based dispersion of the mineral matter according to claim 25, comprising from 0.05% to 5%, by dry weight of at least one hydrosoluble polymer obtained by a process of controlled radical polymerisation using an alcoxyamine of formula (A), relative to the dry weight of the mineral matter.

27. A water-based suspension of a mineral matter, comprising:
water,
at least one mineral matter,
at least one hydrosoluble polymer obtained by a process of controlled radical polymerisation using an alcoxyamine of formula (A), and and possibly at least one grinding aid agent different from the polymer.

28. The water-based suspension of the mineral matter according to claim 27, comprising from 0.05% to 5%, by dry weight of at least one hydrosoluble polymer obtained by a process of controlled radical polymerisation using an alcoxyamine of formula (A), relative to the dry weight of mineral matter.

29. A water-based formulation of a mineral matter, comprising:
water,
at least one mineral matter,
at least one hydrosoluble polymer obtained by a process of controlled radical polymerisation using an alcoxyamine of formula (A), and
possibly at least one dispersing agent and/or at least one grinding aid agent different from the polymer.

30. The water-based formulation of the mineral matter according to claim 29, comprising from 0.05% to 5%, by dry weight of at least one hydrosoluble polymer obtained by a process of controlled radical polymerisation using an alcoxyamine of formula (A), relative to the total weight of the formulation.

31. A paper coating, comprising:
water,
at least one mineral matter,
at least one hydrosolubie polymer obtained by a process of controlled radical polymerisation using an alcoxyamine of formula (A), and
possibly at least one dispersing agent and/or at least one grinding aid agent different from the polymer.

32. The paper coatings according to claim 31, comprising from 0.05% to 5%, by dry weight of at least one hydrosoluble polymer obtained by a process of controlled radical polymerisation using an alcoxyamine of formula (A), relative to the total weight of the formulation.

33. A mass filler, comprising:
water,
at least one mineral matter,
at least one hydrosoluble polymer obtained by a process of controlled radical polymerisation using an alcoxyamine of formula (A), and
possibly at least one dispersing agent and/or at least one grinding aid agent different from the polymer.

34. The mass filler according to claim 33, comprising from 0.05% to 5%, by dry weight of at least one hydrosoluble polymer obtained by a process of controlled radical polymerisation using an alcoxyamine of formula (A), relative to the total weight of the formulation.

35. A water-based paint, comprising:
water,
at least one mineral matter,
at least one hydrosoluble polymer obtained by a process of controlled radical polymerisation using an alcoxyamine of formula (A), and
possibly at least one dispersing agent and/or at least one grinding aid agent different from the polymer.

36. The water-based paint according to claim 35, comprising from 0.05% to 5%, by dry weight of at least one hydrosoluble polymer obtained by a process of controlled radical polymerisation using an alcoxyamine of formula (A), relative to the total weight of the formulation.

37. A cosmetic formulation, comprising:
water,
at least one mineral matter,
at least one hydrosoluble polymer obtained by a process of controlled radical polymerisation using an alcoxyamine of formula (A), and
possibly at least one dispersing agent and/or at least one grinding aid agent different from the polymer.

38. The cosmetic formulation according to claim 37, comprising from 0.05% to 5%, by dry weight of at least one hydrosoluble polymer obtained by a process of controlled radical polymerisation using an alcoxyamine of formula (A), relative to the total weight of the formulation.

39. A dry powder of mineral matter, comprising:
at least one mineral matter,
and at least one hydrosoluble polymer obtained by a process of controlled radical polymerisation using an alcoxyamine of formula (A).

40. The dry powder according to claim 39, comprising from 0.05% to 5%, by dry weight of at least one hydrosoluble polymer obtained by a process of controlled radical polymerisation using an alcoxyamine of formula (A), relative to the total weight of the powder.

41. A dry film obtained from the drying of a water-based formulation of a mineral matter according claim 29, comprising:
at least one mineral matter,
and at least one hydrosoluble polymer obtained by a process of controlled radical polymerisation using an alcoxyamine of formula (A).

42. A filled paper, comprising:
at least one mineral matter,
and at least one hydrosoluble polymer obtained by a process of controlled radical polymerisation using an alcoxyamine of formula (A).

43. A plastic, comprising:
at least one mineral matter,
and at least one hydrosoluble polymer obtained by a process of controlled radical polymerisation using an alcoxyamine of formula (A).
